(19) United States
(12) Patent Application Publication
(10) Pub. No.: US 2006/0254001 A1
(43) Pub. Date: Nov. 16, 2006

(54) Stabilization of hydrogen peroxide during the dissolving of an alkalizing agent in hydrogen peroxide-containing systems

(76) Inventors: Horst Hoeffkes, Duesseldorf (DE); Martina Seiler, Duisburg (DE); Kristin Pauli, Wuppertal (DE)

Correspondence Address: DANN DORFMAN HERRELL AND SKILLMAN;A PROFESSIONAL CORPORATION, 1601 MARKET STREET, SUITE 2400, PHILADELPHIA, PA, 19103-2307, US

(21) Appl. No.: 11/363,478

(22) Filed: Feb. 27, 2006

Related U.S. Application Data
(63) Continuation of application No. PCT/EP04/09207, filed on Aug. 17, 2004.

(30) Foreign Application Priority Data
Aug. 26, 2003 (DE) 103 39 164.9

(51) Int. Cl. A61K 8/00

(52) U.S. Cl. 8/405

(57) ABSTRACT

An alkalization-effecting solid composition that contains, as an agglomerate at least one alkalizing agent and at least one chelating agent. The chelating agents are present along with the alkalizing agent in the same particle of the agglomerate. Hydrogen peroxide decomposition during the dissolution process of alkalization-effecting compositions in the presence of H₂O₂ can be greatly reduced if the composition to be dissolved is this alkalinization-effecting solid composition.

### CROSS-REFERENCE TO RELATED APPLICATIONS

This application is a continuation under 35 U.S.C. §365(c) and 35 U.S.C. §120 of International Application No. PCT/EP2004/009207, filed Aug. 17, 2004. This application also claims priority under 35 U.S.C. §119 of German Patent Application No. 103 39 164.9, filed Aug. 26, 2003. Both the International application and the German application are incorporated by reference in their entireties.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

(1) Field of the Invention

The subject of the present invention is a solid, alkalization-effecting composition that contains at least 75 wt %, based on the weight of the composition, of a mixture of at least one alkalizing agent and at least one chelating agent, as well as the use of this composition to decrease the decomposition of hydrogen peroxide during the dissolution process of solid or pasty components, containing as alkalizing agent the solid, alkalization-effecting composition according to the present invention, the solvent and/or the component to be dissolved containing hydrogen peroxide.

Hydrogen peroxide-containing agents are used in a wide variety of processes, for example in oxidative treatment of any type of fiber, brightening and cleaning of textiles or surfaces, permanent deformation or permanent color change in the context of oxidative coloring or bleaching of fibers, e.g., of keratin-containing fibers.

For bleaching, for example for brightening textiles or bleaching human hair—and in the latter case in particular for “highlighting” application—it is usual to mix solid or pasty preparations having solid oxidizing agents (so-called “bleach boosters”) with a dilute hydrogen peroxide solution immediately before use. This mixture is then applied onto the hair and rinsed out again after a certain contact time.

So-called oxidizing coloring agents are used for permanent, intense coloration of fibers, in particular keratin-containing fibers, with corresponding fastness properties. Such coloring agents usually contain oxidizing dye precursors, so-called developer components, and coupler components. Under the influence of the oxidizing agents added prior to application, the developer components form, among one another or by coupling to one or more coupler components, the actual dyes. Oxidizing coloring agents are characterized by outstanding, long-lasting coloring results.

Temporary coloring results are achieved on a fiber when so-called direct-absorbing dyes are used in coloring agents. Direct-absorbing dyes are inherently colored, and do not require oxidizing agents to produce color. Direct-absorbing dyes can, however, also be used together with oxidizing coloring agents in order to deliberately influence the color shade.

Also known are coloring methods in which direct-absorbing dyes, constituting the only coloring component, bring about a change in color in combination with hydrogen peroxide-containing agents such as, for example, the aforesaid bleaching agents. The hydrogen peroxide-containing agent is responsible for a brightening of the fibers, with the result that the fiber color obtained appears more brilliant than when colored with the direct-absorbing dyes.

The hydrogen peroxide-containing agent is often blended in from separately stored compositions shortly before application, yielding the actual application mixture. This procedure is necessary in particular when the application mixture contains not only hydrogen peroxide but also components that enter into a chemical reaction with hydrogen peroxide. For that reason, the actual application mixture is not stable in storage.

Direct-absorbing dyes, for example, are quite often unstable with respect to hydrogen peroxide. If they are to be used together with hydrogen peroxide, they are mixed shortly before application to yield a hydrogen peroxide-containing composition. Because the oxidizing dye precursors and the direct-absorbing dyes are for the most part solids, they can be added in solid form, as a powder, pellets, or tablets, to the hydrogen peroxide-containing composition shortly before application.

The efficiency, e.g., the brightening power, of the aforesaid bleaching agents, or the coloring power of the aforesaid coloring agents, is greatest at a basic pH, in particular at a pH between 8 and 12. A hydrogen peroxide preparation is, however, not stable in storage at such an alkaline pH. Hydrogen peroxide preparations that are stable in storage possess a neutral, usually acid pH of 2 to 5. In order to arrive at an alkaline application mixture, the component that is to be mixed in contains alkalizing agents.

It is furthermore known that in addition to an alkaline pH, the presence of decomposition accelerators, such as metal cations, zeolites, or bleach boosters, promotes the decomposition of hydrogen peroxide.

Upon the dissolution of solid, alkalization-effecting compositions in an aqueous liquid composition functioning as a solvent, a decomposition of the hydrogen peroxide contained in the liquid and/or the solid composition commonly takes place during the dissolution process. H₂O₂ decomposition is particularly pronounced when the aqueous composition that functions as a solvent is viscous. Such decomposition is accompanied by the evolution of oxygen, and in the worst case becomes apparent as an exothermic chemical reaction. In a medium containing both H₂O₂ and foaming agents, the gas evolution causes foaming. Like the hydrogen peroxide decomposition, this foaming is also undesirable, since the foam interferes with diffusion of the active ingredients into the fibers. If the dissolution process is performed in the usual fashion by agitation in a closed vessel, the result of the gas evolution is excess pressure in that vessel. Upon opening of the vessel after the dissolution process, in the worst case the pressure equalization that simultaneously takes place causes the application mixture to spray in uncontrolled fashion out of the vessel, which can constitute a hazard to the user.

The decomposition of hydrogen peroxide consequently decreases the efficiency of the application mixture and increases the potential hazard when working with hydrogen peroxide-containing agents. It is therefore known to incorporate chelating agents into hydrogen peroxide-containing compositions in order to stabilize the hydrogen peroxide. In the multi-component agents described above, these stabilizers can be a component both of the liquid solvent and of a solid that is to be dissolved. Merely mixing in the chelating agents is not sufficient, however, to achieve a sufficient reduction in hydrogen peroxide decomposition during the dissolution process.

(2) Description of Related Art, Including Information Disclosed Under 37 C.F.R. §§1.97 and 1.98

The document WO-A1-94/03553 relates to solid or liquid bleaching agents that contain hydrogen peroxide, a hydrogen peroxide-releasing substance, and 1,2-ethylenediamine-N,N′-disuccinate or its free acid as an H₂O₂ stabilizer.

The document WO-A1-95/23210 describes solid particles at whose core is a hydrogen peroxide-releasing substance, for example sodium percarbonate or a peroxy-acid, and which are coated with a hydroxycarboxylic acid as a chelating agent. The storage stability of the solid particles is improved by the coating.

### BRIEF SUMMARY OF THE INVENTION

The object of the present invention is, in the context of the production of oxidation-effecting H₂O₂-containing agents made up of at least one liquid component and at least one alkalization-effecting solid or pasty component, to prevent the decomposition of hydrogen peroxide during the dissolution process of the alkalization-effecting solid or pasty component in the liquid component, or to reduce that decomposition greatly as compared with agents of the existing art. In this context, the hydrogen peroxide is contained in at least one of the aforesaid components as an obligatory constituent.

It has now been found, surprisingly, that hydrogen peroxide decomposition during the dissolution process of alkalization-effecting compositions in the presence of H₂O₂ can be greatly reduced if the composition to be dissolved contains, as alkalizing substance, an alkalization-effecting solid composition that contains, as an agglomerate, not only at least one alkalizing agent but also at least one chelating agent. The chelating agents are present along with the alkalizing agent in the same particle of the agglomerate. Alkalization-effecting compositions of this kind are novel.

### BRIEF DESCRIPTION OF THE SEVERAL VIEWS OF THE DRAWING(S)

Not Applicable

### DETAILED DESCRIPTION OF THE INVENTION

A composition is solid for purposes of the invention if it exists as a solid at 20° C. at a pressure of 101,325 Pa.

An agglomerate for purposes of the invention is an agglutination of several different substances forming solid particles.

For purposes of the invention an alkalization-effecting composition is capable, at a concentration of at least 10⁻² mol/l, of raising the pH of an aqueous or aqueous/alcoholic system to a pH greater than 7.

For purposes of the invention, an aqueous system or an aqueous carrier contains at least 10 wt % water. Systems that contain less than 10 wt % water are referred to as “anhydrous.” Aqueous/alcoholic systems or aqueous/alcoholic carriers are understood, for purposes of the present invention, to be aqueous systems containing 3 to 70 wt % of an optionally substituted C₁ to C₄ alcohol having at least one hydroxy group, for example, methoxybutanol, benzyl alcohol, ethyl diglycol or 1,2-propylene glycol, glycerol, and in particular ethanol or isopropanol.

Keratin-containing fibers are understood in the context of this application to be furs, wool, feathers, and in particular human hair.

The term “chelating agent” is familiar to one skilled in the art. The reader is explicitly referred in that connection to the Römpp Chemie Lexikon, 9th expanded and revised edition, Georg Thieme Verlag, Stuttgart (1995), Vol. 1 (A-Cl), page 634.

A first subject of the invention is an alkalization-effecting solid composition that contains
(i) in a quantity of at least 75 wt % based on the weight of the composition, a mixture of (a) at least one particulate alkalizing agent and (b) at least one chelating agent; and
(ii) if applicable, further additives,
   the composition being made up of agglomerates formed from (a), (b) and the optionally contained additives.

The agglomerates of the alkalization-effecting solid composition according to the present invention can be produced in various ways. The alkalizing agents and chelating agents can be utilized in this production process both as solids and as liquids. If two solids are used to produce the agglomerates, they are agglutinated into agglomerates using known press-agglomeration methods. It may be preferred in this context additionally to use so-called binders, for example bentonite, molasses, oils, or waxes. In order to form the agglomerates of the chelating agents and alkalizing agents, the two solid components preferably are first dissolved together in a solvent, for example water, and the solvent is then removed. The solvent is removed using known drying methods, for example a drum-drying method or trickle drying, yielding the alkalization-effecting solid composition according to the present invention.

If either the alkalizing agent or the chelating agent is used as a solid and the other component as a liquid or solution, dispensing of the liquid is to be selected in such a way that a free-flowing powder remains behind after treatment of the solid with the liquid component. Preferably the liquid component is sprayed onto the solid component using a suitable apparatus.

The alkalization-effecting solid compositions obtained as described above can moreover be compacted, extruded, or granulated using usual methods.

The chelating agents can be spatially distributed within the agglomerate in different ways. For example, the chelating agents can be present on the surface of agglomerates of the alkalizing agent as a so-called surface coating. The particles of the alkalizing agent and of the chelating agent can, however, also be present in the agglomerate in uniformly distributed fashion.

The agglomerates preferably have an average particle diameter from 10 to 300 μm, particularly preferably from 100 to 200 μm.

The optional additives can be, in addition to the aforementioned binders, preferably those additives that can be contained in the embodiments defined below of the alkalization-effecting solid composition according to the present invention.

According to the present invention, the usual particulate alkalizing agents known to one skilled in the art, such as the hydroxides, carbonates, hydrogencarbonates, hydroxycarbonates, carbamides, silicates (in particular metasilicates) of ammonium, of alkali metals, and of alkaline-earth metals, as well as alkaline phosphates, can be used. In a preferred embodiment, the alkalization-effecting solid composition according to the present invention contains at least two different particulate alkalizing agents. Mixtures of, for example, a metasilicate and a hydroxycarbonate may be preferred in this context.

In a particularly preferred embodiment, the alkalization-effecting solid composition according to the present invention contains as alkalizing agent at least one metasilicate of ammonium or of the alkali metals or alkaline-earth metals. Very particularly preferred metasilicates according to the present invention are waterglasses that are formed from an aqueous solution of a silicate of the formula (SiO₂)ₙ(Na₂O)ₘ(K₂O)ₚ, where n denotes a positive rational number and m and p, independently of one another, denote a positive rational number or 0, with the provisos that at least one of the parameters m or p is different from 0, and that the ratio between n and the sum of m and p is between 1:4 and 4:1.

Also usable in principle are amorphous sodium silicates having a Na₂O:SiO₂ modulus from 1:2 to 1:3.3, preferably from 1:2 to 1:2.8, and in particular from 1:2 to 1:2.6, which are dissolution-delayed. The dissolution delay as compared with conventional amorphous sodium silicates can be brought about in various ways, for example by surface treatment, compounding, compaction, or overdrying. In the context of this invention, the term “amorphous” is also understood to mean “X-ray amorphous.” This means that in X-ray diffraction experiments, the silicates do not yield sharp X-ray reflections such as those typical of crystalline substances, but instead at most one or more maxima of the scattered X radiation that have a width of several degree units of the diffraction angle. It is nevertheless quite possible to obtain even very good properties if the silicate particles yield blurred or even sharp diffraction maxima in electron diffraction experiments. This is to be interpreted to mean that the products exhibit microcrystalline regions 10 to several hundred nm in size, values up to a maximum of 50 nm and in particular up to a maximum of 20 nm being preferred. Such so-called X-ray amorphous silicates likewise exhibit a dissolution delay with respect to the conventional waterglasses. Compacted amorphous silicates, compounded amorphous silicates, and overdried X-ray amorphous silicates are particularly preferred.

In addition to the components described by the empirical formula, the waterglasses can also contain small quantities of further additives, for example phosphates or magnesium salts.

Waterglasses that are particularly preferred according to the present invention are marketed by, among others, the Henkel company under the designations Ferrosil® 119, Soda waterglass 40/42, Portil® A, Portil® AW, Portil® N and Portil® W, and by PQ Nederlands under the designation Britesil® C.20.

The particulate alkalizing agents are contained in the alkalization-effecting solid compositions according to the present invention preferably in a quantity from 80 to 99.8 wt %, particularly preferably in a quantity from 90 to 98 wt %, in each case based on the weight of the entire composition.

A plurality of chelating agents are known to one skilled in the art from the relevant literature. Particularly suitable are polycarboxylic acids and their water-soluble sodium, potassium, magnesium, and ammonium salts, various fruit acids, derivatives of amino acids, aminopolycarboxylic acids, metaphosphoric, polyphosphoric, and polyphosphonic acids and their salts, alkali-metal stannates such as, for example, sodium stannate, hydroxycarboxylic acids and their salts such as, for example, citric acid, tartaric acid, malic acid, and gluconic acid, glucuronic acid, galactaric acid, as well as benzamides and anilides such as, for example, acetanilide, and the 0-hydroxycarboxylic acids according to WO-A1-95/23210, to which reference is explicitly made.

Examples of polycarboxylic acids according to the present invention are succinic acid, 1,2,3-propanetricarboxylic acid, dipicolinic acid, cyclodextrins, β-alaninediacetic acid and its salts; dihydroxyethyl glycinates, dicarboxymethyl alaninates, tetrahydroxyethyl- and tetrahydroxypropylethylenediamine are also chelating agents according to the present invention.

Further suitable as chelating agents are the water-soluble salts of aminopolycarboxylic acids as well as their sodium, potassium, ammonium, magnesium, calcium, and triethanolamine salts. Aminocarboxylic acids preferably incorporated into the composition according to the present invention of the first subject are ethylenediaminetetraacetic acid (EDTA) and its salts such as, for example, calcium-disodium EDTA, diammonium EDTA, disodium and dipotassium EDTA, triethanolamine EDTA, trisodium and tripotassium EDTA, tetrasodium and tetrapotassium EDTA, as well as nitrilotriacetic acid, hydroxyethylethylenediaminetriacetic acid, cyclohexanediaminetetraacetic acid, diethylenetriaminepentaacetic acid, lauroylethylenediaminetriacetic acid, ethylenediaminedisuccinic acid, and dipicolinic acid, and the corresponding salts. Particularly preferred according to the present invention as an aminopolycarboxylic acid are tetrasodium EDTA and trisodium EDTA, which are marketed, e.g., under the designations Trilon® B and Trilon® A. Further preferred aminocarboxylic acids according to the present invention are 1,2-ethylenediamine-N,N′-diglutaric acid (EDDG) as well as iminodisuccinates such as, for example, 1,2-ethylenediamine-N,N′-disuccinate (EDDS) and 2-hydroxypropylenediamine-N,N′-disuccinate (HPDDS).

Polyphosphoric acids and salts thereof that are preferably used are those of the formula M⁺ₙ₊₂₋ₓ[HₓPₙO₃ₙ₊₁]^{(n+2-x)-}, in which M preferably denotes hydrogen, sodium, or potassium and n is a natural number not equal to zero and 1, and x is a natural number from 0 to 3. Examples of such polyphosphoric acids and their salts according to the present invention are tetrasodium diphosphate, pentasodium triphosphate, and disodium dihydrogendiphosphate.

Also in accordance with the invention are, moreover, the cyclic metaphosphoric acids and their salts having the formula M⁺ₙ[PₙO₃ₙ₊₁]ⁿ⁻, where n is a natural number not equal to zero. An example of a preferred metaphosphoric acid is trisodium metaphosphate or the sodium metaphosphate that is marketed, for example, under the trade name Calgon®.

Examples of polyphosphonic acids according to the present invention are 1-hydroxyethane-1,1-diphosphonic acid (etidronic acid), N,N,N-tri(phosphonomethyl)amine, 1,2-ethylenediaminetetramethylenephosphonic acid (EDTMP), diethylenetriaminepentamethylenephosphonic acid (DTPMP), N,N,N-tri(1-phosphonoethyl)amine, N,N,N-tri(1-phosphonopropyl)amine and N,N,N-tri(2-phosphonoprop-2-yl)amine. Etidronic acid is a preferred polyphosphonic acid.

The chelating agents selected from ethylenediaminetetraacetic acid (EDTA), diethylenetriaminepentaacetic acid, nitriloacetic acid, 1-hydroxyethane-1,1-diphosphonic acid (etidronic acid), dipicolinic acid, acetanilide, and sodium stannate, as well as the physiologically acceptable salts of the aforesaid acids, are particularly preferred.

The chelating agents are preferably contained in these alkalization-effecting compositions according to the present invention in a quantity from 0.1 to 20 wt %, particularly preferably in a quantity from 2 to 10 wt %, in each case based on the weight of the entire composition.

The mixture of alkalizing agents and chelating agents is preferably present as at least 90 wt %, particularly preferably at least 97 wt %, based on the weight of the entire alkalization-effecting solid composition according to the present invention.

The alkalization-effecting solid compositions according to the present invention can contain further cosmetic additives. These additives are those that can be contained in compositions (A), (B), and (C) of the second subject of the invention. Preferred cosmetic additives are surfactants, conditioning ingredients, dyes, and dye precursors. The cosmetic additives are contained in total in the alkalization-effecting solid composition preferably at a proportion from 0 to 25 wt %, particularly preferably from 0 to 10 wt %, very particularly preferably from 0 to 3 wt %, in each case based on the weight of the composition.

A second subject of the present invention is a hydrogen peroxide-containing agent that is obtained by mixing at least two separately manufactured compositions (A) and (B), wherein
(i) composition (A) is anhydrous and contains at least one alkalization-effecting solid composition of the first subject of the invention;
(ii) composition (B) is aqueous or aqueous/alcoholic; and
(iii) at least one of compositions (A) and (B) contains hydrogen peroxide.

The hydrogen peroxide contained in compositions (A) and/or (B) according to the present invention is added to composition (A) or (B), according to the present invention, as a solution or in the form of a solid addition compound of hydrogen peroxide with inorganic or organic compounds such as, for example, sodium perborate, sodium percarbonate, sodium percarbamide, polyvinylpyrrolidone·n H₂O₂ (where n is a positive number greater than 0), urea perhydrate, and melamine perhydrate.

If hydrogen peroxide is a constituent of the anhydrous composition (A), it is present as a dispersed, particulate solid in the form of a solid addition compound of hydrogen peroxide with inorganic or organic compounds. During mixing with composition (B), dissolved hydrogen peroxide is formed by the contact with water. In a preferred embodiment, only one of compositions (A) or (B) contains hydrogen peroxide.

In a preferred embodiment, the alkalization-effecting solid composition according to the present invention is contained as the only alkalizing agent of the hydrogen peroxide-containing agent.

The agent according to the present invention preferably possesses a pH greater than 7.

Composition (A) is preferably solid or pasty. The pasty form can be obtained, for example, by mixing the solid components with oils and/or with liquid and anhydrous nonionogenic surfactants. Preferred oils in this context are, among others, natural and synthetic oils, straight-chain and branched hydrocarbons, and liquid waxes, as well as silicone oils (according to EP-A1-560 088, to whose entire content reference is made, e.g., paraffin oil), dialkyl ethers (such as those disclosed, e.g., in the document DE-A1-196 00 216, to whose entire content reference is made, e.g., di-n-octyl ether and di-n-dodecyl ether), and esters of carboxylic acids and of carbon dioxide.

Furthermore, composition (A) contains the alkalization-effecting solid composition according to the present invention preferably in a quantity from 1 to 40 wt %, particularly preferably in a quantity from 2 to 30 wt %, in each case based on the weight of the entire composition (A).

Composition (A) preferably contains at least one bleach booster. Solid peroxo-compounds that do not represent addition products of hydrogen peroxides with other components are particularly advantageous for the bleaching of keratin-containing fibers. Selection of the peroxo- compounds contained in composition (A) according to the present invention is not subject, in principle, to any limitations; usual peroxo-compounds known to one skilled in the art are, for example, (i) peroxodisulfates, persulfates, and peroxodiphosphates such as, for example, ammonium peroxodisulfate, potassium peroxodisulfate, sodium peroxodisulfate, ammonium persulfate, potassium persulfate, sodium persulfate, potassium peroxodiphosphate; and furthermore (ii) peroxides of the alkali and alkaline-earth metals, such as magnesium and barium peroxide; as well as (iii) peroxocarboxylic acids or their physiologically acceptable salts, for example magnesium perphthalate. Among these peroxo-compounds, which can also be used in combination, the inorganic compounds are preferred according to the present invention. The peroxodisulfates, in particular ammonium peroxodisulfate, are particularly preferred.

Additionally usable as bleach boosters, in particular for bleaching or washing textile fibers of any kind, are compounds that, under perhydrolysis conditions, yield aliphatic peroxocarboxylic acids having preferably 1 to 10 carbon atoms, in particular 2 to 4 carbon atoms, and/or optionally substituted perbenzoic acids. Substances that carry O— and/or N-acyl groups having the aforesaid number of carbon atoms, and/or optionally substituted benzoyl groups, are suitable. Multiply acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic acid anhydrides, in particular phthalic acid anhydride, acylated polyvalent alcohols, in particular triacetin, ethylene glycol diacetate, and 2,5-diacetoxy-2,5-dihydrofuran, are preferred.

Further preferred bleach activators are (iv) cationic nitriles, in particular of the formula below: 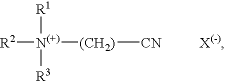
in which R¹ denotes —H, —CH₃, a C₂₋₂₄ alkyl or alkenyl radical, a substituted C₂₋₂₄alkyl or alkenyl radical having at least one substituent from the group —Cl, —Br, —OH, —NH₂, —CN, an alkyl or alkenylaryl radical having a C₁₋₂₄ alkyl group, or a substituted alkyl or alkenylaryl radical having a C₁₋₂₄ alkyl group and at least one further substituent on the aromatic ring, R² and R³ are selected, independently of one another, from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—CH(OH)—CH₃, —CH(OH)—CH₂—CH₃, —(CH₂CH₂—O)ₙH where n=1, 2, 3, 4, 5 or 6, and X is an anion.

This general formula covers a plurality of cationic nitriles that are usable in the context of the present invention. It is particularly advantageous if the compositions according to the present invention contain cationic nitriles in which R¹ denotes methyl, ethyl, propyl, isopropyl or an n-butyl, n-hexyl, n-octyl, n-decyl, n-dodecyl, n-tetradecyl, n-hexadecyl or n-octadecyl radical. R² and R³ are preferably selected from methyl, ethyl, propyl, isopropyl and hydroxyethyl; one or both radicals can advantageously also be a cyanomethylene radical.

The bleach boosters are contained in composition (A) according to the present invention preferably in quantities from 5 to 60 wt %, in particular in quantities from 8 to 30 wt %, in each case based on the weight of the entire composition (A).

Composition (B) is an aqueous or aqueous/alcoholic system. It can be present in the form of an emulsion, e.g., a W/O or O/W emulsion. The viscosity of composition (B) preferably falls in a range from 1 to 100,000 mPa·s, preferably from 1,000 to 70,000 mPa·s, particularly preferably from 6,000 to 50,000 mPa·s, and very particularly preferably from 10,000 to 30,000 mPa·s. The viscosities are measured with a Brookfield RVT viscosimeter at a temperature of 20° C. at 4 rpm using a no. 4 spindle. The spindle for measuring the aforesaid viscosities is preferably selected, however, on the basis of the viscosity range (measured under the aforementioned experimental conditions) in accordance with TABLE 1:

TABLE 1

| Spindle no. | Viscosity range (mPa · s) |
|---|---|
| 1 | up to 2,500 |
| 2 | >2,500 to 10,000 |
| 3 | >10,000 to 25,000 |
| 4 | >25,000 to 50,000 |
| 5 | >50,000 to 100,000 |

In a specific embodiment, composition (B) has a viscosity from 1 to 50,000 mPa·s, particularly preferably from 500 to 25,000 mPa·s, very particularly preferably from 500 to 15,000 mPa·s. The viscosities of this specific embodiment are measured with a Brookfield RVT viscosimeter at 20° C. and 20 rpm, using a no. 4 spindle.

Composition (B) preferably possesses a pH in a range from pH 2 to 7, particularly preferably in a range from pH 3 to 6.

Composition (B) can additionally contain at least one chelating agent. The aforementioned chelating agents are considered preferred chelating agents.

Compositions (A) and (B) can additionally contain at least one surfactant, both anionic as well as zwitterionic, ampholytic, nonionic, and cationic surfactants being suitable in principle. It has proven advantageous in many cases, however, to select the surfactants from anionic, zwitterionic, or nonionic surfactants.

All anionic surface-active substances suitable for use on the human body are appropriate as anionic surfactants in preparations according to the present invention. These substances are characterized by an anionic group imparting water solubility, for example a carboxylate, sulfate, sulfonate, or phosphate group, and a lipophilic alkyl group having approximately 10 to 22 carbon atoms. Glycol or polyglycol ether groups, ester, ether, and amide groups, and hydroxyl groups can also be contained in the molecule. Examples of suitable anionic surfactants are, in each case in the form of the sodium, potassium, and ammonium and mono-, di-, and trialkanolammonium salts having two or three carbon atoms in the alkanol group:
linear fatty acids having 10 to 22 carbon atoms (soaps);
ethercarboxylic acids of the formula R—O—(CH₂—CH₂O)ₓ—CH₂—COOH, in which R is a linear alkyl group having 10 to 22 carbon atoms and x=0 or is 1 to 16;
acylsarcosides having 10 to 18 carbon atoms in the acyl group;
acyltaurides having 10 to 18 carbon atoms in the acyl group;
acylisethionates having 10 to 18 carbon atoms in the acyl group;
sulfosuccinic acid mono- and -dialkyl esters having 8 to 18 carbon atoms in the alkyl group and sulfosuccinic acid monoalkylpolyoxyethyl esters having 8 to 18 carbon atoms in the alkyl group and 1 to 6 oxyethyl groups;
linear alkanesulfonates having 12 to 18 carbon atoms;
linear alpha-olefinsulfonates having 12 to 18 carbon atoms;
alpha-sulfofatty acid methyl esters of fatty acids having 12 to 18 carbon atoms;
alkyl sulfates and alkylpolyglycol ether sulfates of the formula R—O(CH₂—CH₂O)ₓ—SO₃H, in which R is a preferably linear alkyl group having 10 to 18 carbon atoms and x=0 or is 1 to 12;
mixtures of surface-active hydroxysulfonates according to DE-A-37 25 030;
sulfated hydroxyalkylpolyethylene and/or hydroxyalkylenepropylene glycol ethers according to DE-A-37 23 354;
sulfonates of unsaturated fatty acids having 12 to 24 carbon atoms and 1 to 6 double bonds, according to DE-A-39 26 344;
esters of tartaric acid and citric acid with alcohols that represent addition products of approximately 2 to 15 molecules of ethylene oxide and/or propylene oxide with fatty alcohols having 8 to 22 carbon atoms.

Preferred anionic surfactants are alkyl sulfates, alkylpolyglycol ether sulfates, and ethercarboxylic acids having 10 to 18 carbon atoms in the alkyl group and up to 12 glycol ether groups in the molecule, and in particular salts of saturated and, in particular, unsaturated C₈-C₂₂ carboxylic acids, such as oleic acid, stearic acid, isostearic acid, and palmitic acid.

Those surface-active compounds that contain in the molecule at least one quaternary ammonium group and at least one —COO⁽⁻⁾ or —SO₃⁽⁻⁾ group are referred to as zwitterionic surfactants. Particularly suitable zwitterionic surfactants are the so-called betaines, such as the N-alkyl-N,N-dimethylammonium glycinates, for example cocalkyldimethylammonium glycinate, N-acylaminopropyl-N,N-dimethylammonium glycinates, for example cocacylaminopropyldimethylammonium glycinate, and 2-alkyl-3-carboxymethyl-3-hydroxyethylimidazolines respectively having 8 to 18 carbon atoms in the alkyl or acyl group, as well as cocacylaminoethylhydroxyethylcarboxymethyl glycinate. A preferred zwitterionic surfactant is the fatty acid amide derivative known by the INCI name Cocamidopropyl betaine.

Ampholytic surfactants are understood to be those surface-active compounds that contain in the molecule, in addition to a C₈₋₈ alkyl or acyl group, at least one free amino group and at least one —COOH— or —SO₃H group, and are suitable for the formation of internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids, and alkylaminoacetic acids, respectively having approximately 8 to 18 carbon atoms in the alkyl group. Particularly preferred ampholytic surfactants are N-cocalkylaminopropionate, cocacylaminoethylaminopropionate, and C₁₂₋₁₈ acylsarcosine.

Nonionic surfactants contain as a hydrophilic group, for example, a polyol group, a polyalkylene glycol ether group, or a combination of a polyol and polyglycol ether group. Such compounds are, for example:
addition products of 2 to 30 mol ethylene oxide and/or 0 to 5 mol propylene oxide with linear fatty alcohols having 8 to 22 carbon atoms, with fatty acids having 12 to 22 carbon atoms, and with alkylphenols having 8 to 15 carbon atoms in the alkyl group;
C₁₂₋₂₂ fatty acid mono- and -diesters of addition products of 1 to 30 mol ethylene oxide with glycerol;
C₈₋₂₂ alkyl mono- and -oligoglycosides and their ethoxylated analogs;
addition products of 5 to 60 mol ethylene oxide with castor oil and hardened castor oil;
addition products of ethylene oxide with sorbitan fatty acid esters;
addition products of ethylene oxide with fatty acid alkanolamides.

Examples of cationic surfactants usable in the compositions according to the present invention are, in particular, quaternary ammonium compounds. Ammonium halides such as alkyltrimethylammonium chlorides, dialkyldimethylammonium chlorides, and trialkylmethylammonium chlorides, e.g., cetyltrimethylammonium chloride, stearyltrimethylammonium chloride, distearyldimethylammonium chloride, lauryldimethylammonium chloride, lauryldimethylbenzylammonium chloride, and tricetylmethylammonium chloride are preferred. The quaternized protein hydrolysates represent further cationic surfactants usable according to the present invention.

Likewise suitable according to the present invention are cationic silicone oils such as, for example, the commercially available products Q2-7224 (manufacturer: Dow Corning; a stabilized trimethylsilylamodimethicone), Dow Corning 929 Emulsion (containing a hydroxylamino-modified silicone that is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80).

Alkylamidoamines, in particular fatty acid amidoamines such as the stearylamidopropyldimethylamine obtainable under the name Tego Amid®S 18, are characterized not only by a good conditioning effect but especially by their good biodegradability.

Also highly biodegradable are quaternary ester compounds—so-called “esterquats”—such as the methylhydroxyalkyldialkoyloxyalkylammonium methosulfates marketed under the trade name Stepantex®, and the products marketed under the trade name Dehyquart®, such as Dehyquart® AU-46.

An example of a quaternary sugar derivative usable as a cationic surfactant is represented by the commercial product Glucquat® 100, which according to INCI nomenclature is a “Lauryl methyl gluceth-10 hydroxypropyl dimonium chloride”.

The respective compounds having alkyl groups that are used as surfactants can each be uniform substances. It is generally preferred, however, to proceed from natural vegetable or animal raw materials when producing these substances, so that substance mixtures having different alkyl chain lengths (depending on the particular raw material) are obtained.

In the case of the surfactants that represent addition products of ethylene oxide and/or propylene oxide with fatty alcohols, or derivatives of these addition products, both products having a “normal” homolog distribution and those having a restricted homolog distribution can be used. A “normal” homolog distribution is understood to mean mixtures of homologs that are obtained upon the reaction of fatty alcohol and alkylene oxide using alkali metals, alkali-metal hydroxides, or alkali-metal alcoholates as catalysts. Restricted homolog distributions, on the other hand, are obtained when, for example, hydrotalcites, alkaline-earth metal salts of ethercarboxylic acids, or alkaline-earth metal oxides, hydroxides, or alcoholates are used as catalysts. The use of products having a restricted homolog distribution can be preferred.

Compositions (A) and/or (B) can preferably additionally contain a conditioning ingredient selected from the group that is constituted by cationic surfactants, cationic polymers, alkylamidoamines, paraffin oils, vegetable oils, and synthetic oils.

Cationic polymers can preferably be used as conditioning ingredients. These are, as a rule, polymers that contain a quaternary nitrogen atom, for example in the form of an ammonium group.

Preferred cationic polymers are, for example:
quaternized cellulose derivatives such as those commercially available under the designations Celquat® and Polymer JR®. The compounds Celquat® H 100, Celquat® L 200, and Polymer JR®400 are preferred quaternized cellulose derivatives;
polymeric dimethyldiallylammonium salts and their copolymers with acrylic acid, as well as esters and amides of acrylic acid and methacrylic acid. The products available commercially under the designations Merquat®100 (poly(dimethyldiallylammonium chloride)), Merquat®550 (dimethyidiallylammonium chloride/acrylamide copolymer), and Merquat®280 (dimethyldiallylammonium chloride/acrylic acid copolymer) are examples of such cationic polymers;
copolymers of vinylpyrrolidone with quaternized derivatives of dialkylaminoacrylate and -methacrylate, for example vinylpyrrolidone/dimethylaminomethacrylate copolymers quaternized with diethylsulfate. Such compounds are obtainable commercially under the designations Gafquat®734 and Gafquat®755;
vinylpyrrolidone/methoimidazolinium chloride copolymer, such as those sold under the designation Luviquat®;
quaternized poly(vinylalcohol);
   and the polymers known under the designations
polyquaternium-2,
polyquaternium-17,
polyquaternium-18, and
polyquaternium-27, having quaternary nitrogen atoms in the main polymer chain.

Cationic polymers of the first four above-mentioned groups are particularly preferred; polyquaternium-2, polyquaternium-10, and polyquaternium-22 are very particularly preferred.

Silicone oils are additionally suitable as conditioning ingredients, in particular dialkyl- and alkylarylsiloxanes such as, for example, dimethylpolysiloxane and methylphenylpolysiloxane, as well as their alkoxylated and quaternized analogs. Examples of such silicones are the products marketed by Dow Corning under the designations DC 190, DC 200, DC 344, DC 345, and DC 1401, and the commercial products Q2-7224 (manufacturer: Dow Corning; a stabilized trimethylsilylamodimethicone), Dow Corning® 929 Emulsion (containing a hydroxylamino-modified silicone that is also referred to as amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxanes, Quaternium-80).

Also usable as conditioning ingredients are paraffin oils, synthetically produced oligomeric alkenes, and vegetable oils such as jojoba oil, sunflower oil, orange oil, almond oil, wheat germ oil, and peach-kernel oil.

Hair-conditioning compounds that are also suitable are phospholipids, for example soy lecithin, egg lecithin, and kephalins.

In a further embodiment, compositions (A) and/or (B) according to the present invention additionally contain structure-improving ingredients. Hair structure-improving ingredients of this kind are represented by vitamins and their derivatives and precursors. Particularly preferred according to the present invention are panthenol and its physiologically acceptable derivatives. Such derivatives are, in particular, the esters and ethers of panthenol as well as cationically derivatized panthenols. Individual representatives are, for example, panthenol triacetate, panthenol monoethyl ether and its monoacetate, and the cationic panthenol derivatives disclosed in WO 92/13829 A1. A further panthenol derivative preferred according to the present invention is its precursor pantolactone. Within this group, panthenol is preferred. A further example of a structure-improving vitamin is pyridoxine (vitamin B6).

Polyvinylpyrrolidone (PVP) is also known, and is preferred according to the present invention, for its fiber structure-improving properties.

Further structure-improving compounds that are particularly effective according to the present invention are represented by the aldehydes. Particularly preferred examples are formaldehyde and formaldehyde-cleaving compounds, for example methoxymethyl ester, dimethylol (thio)urea derivatives, oxazolidine derivatives, N-hydroxymethylmaleinimide, hexamethylenetetramine and its derivatives, hydantoin derivatives, pyridinium-substituted dimethyl ethers, imidazolidinyl urea derivatives, isothiazolinones, 2-bromo-2-nitropropanediol, and 5-bromo-5-nitro-1,3-dioxane. Other particularly preferred aldehydes are acetaldehyde, glyoxal, glyceraldehyde, and glutaric dialdehyde.

A further suitable group of structure-improving ingredients are plant extracts.

These extracts are usually produced by the extraction of whole plants. In individual cases, however, it may also be preferred to produce the extracts exclusively from blossoms and/or leaves of the plant.

With regard to the plant extracts usable according to the present invention, reference is made in particular to the extracts that are listed in the table beginning on page 44 of the 3rd edition of the Guideline for declaring the contents of cosmetic agents [Leiffaden zur Inhaltsstoffdeklaration kosmetischer Mittel], published by the Association of the personal hygiene and washing agents industry [Industrieverband Korperpflege- und Waschmittel e.V. (IKW)], Frankfurt.

According to the present invention, the extracts from oak bark, nettle, hamamelis, hops, chamomile, burdock root, horsetail, hawthorn, linden blossom, almonds, aloe vera, pine needles, horse chestnut, sandalwood, juniper, coconut, mango, apricot, lemon, wheat, kiwi fruit, melon, orange, grapefruit, salvia, rosemary, birch, mallow, lady's-smock, wild thyme, yarrow, thyme, lemon balm, restharrow, coltsfoot, hibiscus, meristem, green tea, ginseng, and ginger root are especially preferred.

The extracts from oak bark, nettle, hamamelis, hops, chamomile, burdock root, horsetail, linden blossom, almonds, aloe vera, coconut, mango, apricot, lemon, wheat, kiwi fruit, melon, orange, grapefruit, salvia, rosemary, birch, lady's-smock, wild thyme, yarrow, restharrow, meristem, green tea, ginseng, and ginger root are particularly preferred.

The extracts from almonds, aloe vera, coconut, mango, apricot, lemon, wheat, kiwi fruit, melon, and green tea are very particularly suitable for the compositions according to the present invention.

Water, alcohols, and mixtures thereof can be used as extraction agents for producing the aforesaid plant extracts. Among the alcohols, lower alcohols such as ethanol and isopropanol, but in particular polyvalent alcohols such as ethylene glycol and propylene glycol, both as sole extraction agents and mixed with water, are preferred. Plant extracts based on water/propylene glycol at a ratio from 1:10 to 10:1 have proven particularly suitable.

According to the present invention the plant extracts can be used in both pure and diluted form. If they are used in diluted form, they usually contain approximately 2 to 80 wt % active substance, and contain as the solvent the extraction agent or extraction agent mixture used to obtain them.

It may furthermore be preferred to use in the compositions according to the present invention mixtures of several, in particular two, different plant extracts.

Honey extracts are likewise preferred according to the present invention as structure-improving ingredients. These extracts are obtained in a manner analogous to the plant extracts, and usually contain 1 to 10 wt %, in particular 3 to 5 wt %, of active substance. Water/propylene glycol mixtures can be preferred extraction agents here as well.

Further structure-improving ingredients are protein hydrolysates, in particular elastin, collagen, keratin, milk protein, soy protein, almond protein, and wheat protein hydrolysates, condensation products thereof with fatty acids, and quaternized protein hydrolysates. Highly degraded keratin hydrolysates having molar weights in the range from 400 to 800 are particularly preferred. Also particularly preferred according to the present invention are quaternized protein hydrolysates such as those marketed, for example, under the commercial designations Gluadin® WQ (INCI name: Laurdimonium hydroxypropyl hydrolyzed wheat protein) and Crotein® Q (INCI name: Hydroxypropyltrimonium hydrolyzed collagen).

In addition to the quaternized protein hydrolysates, quaternary polymers also represent structure-improving compounds that are preferred according to the present invention. The polymers that are marketed under the commercial designations Mirapol® A15 (INCI name: polyquaternium-2), Onamer® M (INCI name: Polyquaternium-1), and Merquat® 100 (INCI name: Polyquaternium-6) are particularly preferred.

Additional fiber structure-improving ingredients are mono-, di-, and oligosaccharides such as, for example, glucose, galactose, fructose, fruit sugars, sucrose, and lactose. Derivatives of these pentoses and hexoses, such as the corresponding -onic and -uronic acids (sugar acids), sugar alcohols, sugar amines, for example N-glucosamine, and glycosides, can also be used according to the present invention. The sugar acids can be used according to the present invention in free form, in the form of their salts (calcium, magnesium, and zinc salts are preferred), and in the form of their esters or lactones. Preferred sugar acids are gluconic acid, gluconic acid gamma-lactone, lactobionic acid, glucuronic acid and its mono- and dilactones, pangamic acid, saccharic acid, mannosaccharic acid and its mono- and dilactones, as well as mucic acid and its mono- and dilactones. Preferred sugar alcohols are sorbitol, mannitol, and dulcitol. Preferred glycosides are the methylglucosides. Of this group, glucose, N-glucosamine, and gluconic acid are particularly preferred.

Certain amino acids are also usable as hair structure-improving ingredients in the context of the present invention. Examples are the amino acids serine, threonine, and tyrosine described in DE-195 22 569, to which reference is expressly made here. Also preferred according to the present invention are derivatives of serine, for example serine phosphate. A further structure-improving amino acid is represented by lysine. Serine is a particularly preferred fiber structure-improving ingredient.

Certain acids, in particular a-hydroxycarboxylic acids, and their salts, can also be used to improve structure. Structure-improving acids preferred according to the present invention are lactic acid, malic acid, tartaric acid, glyceric acid, and maleic acid. Lactic acid is particularly preferred. Specific phosphonic acids and their salts also improve the structure of keratin-containing fibers. Phosphonic acids preferred according to the present invention are n-octylphosphonic acid and n-decylphosphonic acid.

Lipid-soluble ester alcohols or ester polyols are additionally known for their structure-improving effect. They are to be regarded as lipid-soluble when 5 wt % of these products dissolve to clarity in cetyl alcohol at 80° C.

The ester alcohols or ester polyols that are suitable according to the present invention are obtainable by reacting an epoxy fatty acid with water or with a univalent or polyvalent alcohol having 1 to 10 carbon atoms, opening the epoxide ring and forming an adjacent dihydroxylethyl or hydroxylalkoxyethyl group. The epoxy fatty acid can also be an epoxidation product of a technical-grade fatty acid ester having a saturated fatty-acid content. The epoxide oxygen content should, however, be at least 3 wt %, preferably 5 to 10 wt %.

The epoxy fatty acid esters are either epoxidated fatty acid esters of univalent alcohols, i.e., for example, epoxidated oleic acid methyl ester, linoleic acid methyl ester, ricinoleic acid methyl ester, or epoxidated fatty acid esters of polyvalent alcohols, e.g., glycerol monooleate or propylene glycol monooleate, or epoxidated fatty acid triglycerides, e.g., oleic acid triglyceride, or unsaturated oils such as e.g., olive oil, soybean oil, sunflower oil, linseed oil, colza oil.

Unsaturated fatty acid methyl ester epoxides from unsaturated vegetable fatty acids, principally, are of particular technical interest. Particularly preferred as an ester polyol, therefore, is the reaction product of a vegetable oil fatty acid methyl ester epoxidate with a polyol having 2-6 carbon atoms and 2-6 hydroxyl groups. Ethylene glycol, 1,2-propylene glycol, 1,3-propylene glycol, butanediol, pentanediol, hexanediol, glycerol, trimethylolpropane, pentaerythritol, sorbitol, or diglycerol, for example, can be present as polyols.

Particularly well suited for the compositions according to the present invention as an ester polyol is the reaction product of a vegetable fatty acid methyl ester epoxidate with trimethylpropane, having a hydroxyl number from 350 to 450. A product of this kind based on soybean oil fatty acid methyl ester epoxide and trimethylolpropane is available under the commercial designation Sovermol®760.

Vitamin B₃ can also be used as a structure-improving ingredient. The compounds nicotinic acid and nicotinic acid amide (niacinamide) are often listed under this designation. Nicotinic acid amide is preferred according to the present invention.

Vitamin H is also usable as a structure-improving ingredient for purposes of the present invention. “Vitamin H” is the designation for the compound (3aS,4S, 6aR)-2-oxohexahydrothienol[3,4-d]-imidazole-4-valerianic acid, for which, however, the common name “biotin” has now become established.

Structure-improving ingredients that are particularly preferred according to the present invention are selected from panthenol, physiologically acceptable panthenol derivatives, mono-, di-, and oligosaccharides, serine, glyceric acid, vitamin B6, niacinamide, polyvinylpyrrolidone, gluconic acid, biotin, and the lipid-soluble ester alcohols or ester polyols.

The compositions according to the present invention contain the structure-improving ingredients preferably in quantities from 0.1 to 5 wt %, particularly preferably in quantities from 0.2 to 2 wt %.

In a preferred embodiment of the present invention, the compositions furthermore contain a magnesium compound. The compositions according to the present invention can be further optimized in terms of their structure-maintaining properties by the addition of Mg²⁺ cations. Preferred magnesium compounds are inorganic and organic Mg²⁺ salts such as, for example, the halides, carbonates and hydrogencarbonates, the acetate and the citrate. If a coloring agent is being formulated, compositions (A) or (B) according to the present invention contain:
at least one direct-absorbing dye and/or
at least one dye precursor product of the developer type (developer component), as well as, if applicable, at least one dye precursor product of the coupler type (coupler component).

The nitro dyes have proven to be suitable as direct-absorbing dyes. According to the present invention, “nitro dyes” is understood to mean the coloring components that comprise at least one aromatic ring system that carries at least one nitro group.

Particularly preferred nitro dyes are HC Yellow 2, HC Yellow 4, HC Yellow 5, HC Yellow 6, HC Yellow 12, HC Orange 1, HC Red 1, HC Red 3, HC Red 10, HC Red 11, HC Red 13, HC Red BN, HC Blue 2, HC Blue 12, HC Violet 1, as well as 1,4-diamino-2-nitrobenzene, 2-amino-4-nitrophenol, 1,4-bis-(β-hydroxyethyl)amino-2-nitrobenzene, 3-nitro-4-(β-hydroxyethyl)aminophenol, 2-(2′-hydroxyethyl)amino-4,6-dinitrophenol, 1-(2′-hydroxyethyl)amino-4-methyl-2-nitrobenzene, 1-amino-4-(2′-hydroxyethyl)-amino-5-chloro-2-nitrobenzene, 4-amino-3-nitrophenol, 1-(2′-ureidoethyl)amino-4-nitrobenzene, 4-amino-2-nitrodiphenylamine-2′-carboxylic acid, 6-nitro-1,2,3,4-tetrahydroquinoxaline, picramic acid and its salts, 2-amino-6-chloro-4-nitrophenol, 4-ethylamino-3-nitrobenzoic acid, and 2-chloro-6-ethylamino-1-hydroxy-4-nitrobenzene.

In addition to the nitro dyes, the azo dyes, anthraquinone, or naphthoquinone are also synthetic direct-absorbing dyes preferred according to the present invention. Preferred direct-absorbing dyes of this type are, for example, Disperse Orange 3, Disperse Blue 3, Disperse Violet 1, Disperse Violet 4, Acid Violet 43, Disperse Black 9, and Acid Black 52, as well as 2-hydroxy-1,4-naphthoquinone.

It may additionally be preferred according to the present invention if the synthetic direct-absorbing dye carries a cationic group. The following are particularly preferred:
(i) cationic triphenylmethane dyes,
(ii) aromatic systems that are substituted with a quaternary nitrogen group, and
(iii) direct-absorbing dyes which contain a heterocycle that comprises at least one quaternary nitrogen atom.

Examples of dyes of class (i) are, in particular, Basic Blue 7, Basic Blue 26, Basic Violet 2, and Basic Violet 14.

Examples of dyes of class (ii) are, in particular, Basic Yellow 57, Basic Red 76, Basic Blue 99, Basic Brown 16, and Basic Brown 17.

Examples of dyes of class (iii) are disclosed in particular in EP-A2-998 908, to which reference is explicitly made at this point, in claims 6 to 11.

Preferred cationic direct-absorbing dyes of group (iii) are, in particular, the following compounds:

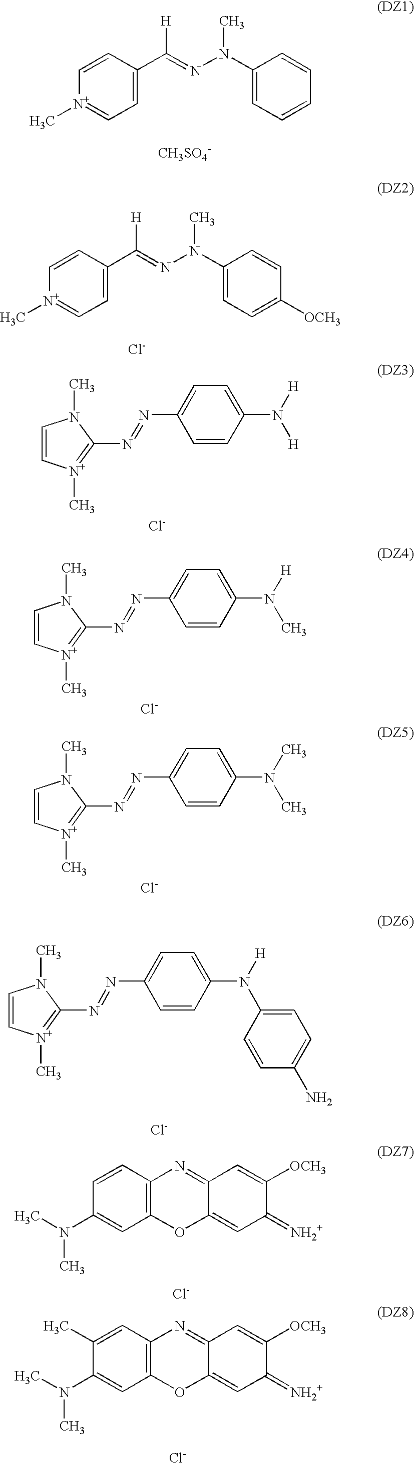

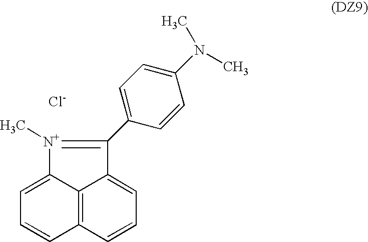

The compounds of formulas (DZ1), (DZ3), and (DZ5) are very particularly preferred cationic direct-absorbing dyes of group (iii).

Compositions (A) or (B) according to the present invention can furthermore also contain naturally occurring dyes, for example those contained in red henna, neutral henna, black henna, chamomile blossoms, sandalwood, black tea, buckthorn bark, salvia, logwood, madder root, catechu, Spanish cedar, and alkanna root.

Compositions (A) or B) according to the present invention contain the direct-absorbing dyes preferably in a quantity from 0.01 to 20 wt %, in each case based on the total weight of the respective composition containing the direct-absorbing dye.

Primary aromatic amines having a further free or substituted hydroxy or amino group located in the para- or ortho- position, diaminopyridine derivatives, heterocyclic hydrazones, 4-aminopyrazole derivatives, and 2,4,5,6-tetraaminopyrimidine and derivatives thereof, are usually used as developer components.

It may be preferred according to the present invention to use as a developer component a p-phenylenediamine derivative or one of its physiologically acceptable salts. Particularly preferred are p-phenylenediamine derivatives of formula (E1) 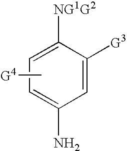
in which
G¹ denotes a hydrogen atom, a C₁ to C₄ alkyl radical, a C₁ to C₄ monohydroxyalkyl radical, a C₂ to C₄ polyhydroxyalkyl radical, a (C₁ to C₄) alkoxy-(C₁ to C₄) alkyl radical, a 4′-aminophenyl radical, or a C₁ to C₄ alkyl radical that is radically substituted with a nitrogen-containing group, with a phenyl radical, or with a 4′-aminophenyl radical;
G² denotes a hydrogen atom, a C₁ to C₄ alkyl radical, C₁ to C₄ monohydroxyalkyl radical, a C₂ to C₄ polyhydroxyalkyl radical, a (C₁ to C₄) alkoxy-(C₁ to C₄) alkyl radical or a C₁ to C₄ alkyl radical that is substituted with a nitrogen-containing group;
G³ denotes a hydrogen atom, a halogen atom such as a chlorine, bromine, iodine, or fluorine atom, a C₁ to C₄ alkyl radical, a C₁ to C₄ monohydroxyalkyl radical, a C₂ to C₄ polyhydroxyalkyl radical, a C₁ to C₄ hydroxyalkoxy radical, a C₁ to C₄ acetylaminoalkoxy radical, a C₁ to C₄ mesylaminoalkoxy radical, or a C₁ to C₄ carbamoylaminoalkoxy radical;
G⁴ denotes a hydrogen atom, a halogen atom, or a C₁ to C₄ alkyl radical; or
if G³ are G⁴ are in the ortho-position with respect to one another, they can together form a bridging α,ω-alkylenedioxo group, for example an ethylenedioxy group.

Examples of the C₁ to C₄ alkyl radicals mentioned as substituents in the compounds according to the present invention are the methyl, ethyl, propyl, isopropyl and butyl groups. Ethyl and methyl are preferred alkyl radicals. C₁ to C₄ alkoxy radicals preferred according to the present invention are, for example, a methoxy or an ethoxy group. A hydroxymethyl, 2-hydroxyethyl, 3-hydroxypropyl, or 4-hydroxybutyl group may also be cited as preferred examples of a C₁ to C₄ hydroxyalkyl group. A 2-hydroxyethyl group is particularly preferred. A particularly preferred C₂ to C₄ polyhydroxyalkyl group is the 1,2-dihydroxyethyl group. Examples of halogen atoms are, according to the present invention, F, Cl, or Br atoms; Cl atoms are very particularly preferred. The additional terms used are derived, according to the present invention, from the definitions given here. Examples of nitrogen-containing groups of formula (E1) are, in particular, the amino groups, C₁ to C₄ monoalkylamino groups, C₁ to C₄ dialkylamino groups, C₁ to C₄ trialkylammonium groups, C₁ to C₄ monohydroxyalkylamino group, imidazolinium, and ammonium.

Particularly preferred p-phenylenediamines of formula (E1) are selected from p-phenylenediamine, p-toluylenediamine, 2-chloro-p-phenylenediamine, 2,3-dimethyl-p-phenylenediamine, 2,6-dimethyl-p-phenylenediamine, 2,6-diethyl-p-phenylenediamine, 2,5-dimethyl-p-phenylenediamine, N,N-dimethyl-p-phenylenediamine, N,N-diethyl-p-phenylenediamine, N,N-dipropyl-p-phenylenediamine, 4-amino-3-methyl-(N,N-diethyl)aniline, N,N-bis-(β-hydroxyethyl)-p-phenylenediamine, 4-N,N-bis-(β-hydroxyethyl)amino-2-methylaniline, 4-N,N-bis-(β-hydroxyethyl)amino-2-chloroaniline, 2-(β-hydroxyethyl)-p-phenylenediamine, 2-(α,β-dihydroxyethyl)-p-phenylenediamine, 2-fluoro-p-phenylenediamine, 2-isopropyl-p-phenylenediamine, N-(β-hydroxypropyl)-p-phenylenediamine, 2-hydroxymethyl-p-phenylenediamine, N,N-dimethyl-3-methyl-p-phenylenediamine, N,N-(ethyl,β-hydroxyethyl)-p-phenylenediamine, N-(β,γ-dihydroxypropyl)-p-phenylenediamine, N-(4′-aminophenyl)-p-phenylenediamine, N-phenyl-p-phenylenediamine, 2-(β-hydroxyethyloxy)-p-phenylenediamine, 2-(β-acetylaminoethyloxy)-p-phenylenediamine, N-(β-methoxyethyl)-p-phenylenediamine, and 5,8-diaminobenzo-1,4-dioxane, as well as their physiologically acceptable salts.

p-Phenylenediamine derivatives of formula (E1) that are very particularly preferred according to the present invention are p-phenylenediamine, p-toluylenediamine, 2-(β-hydroxyethyl)-p-phenylenediamine, 2-(α,β-dihydroxyethyl)-p-phenylenediamine, and N,N-bis-(β-hydroxyethyl)-p-phenylenediamine.

It may furthermore be preferred according to the present invention to use as developer components compounds that contain at least two aromatic nuclei that are substituted with amino and/or hydroxyl groups.

Among the binuclear developer components usable in compositions (A) or (B) according to the invention may be cited, in particular, those compounds that correspond to formula (E2) below, as well as their physiologically acceptable salts: 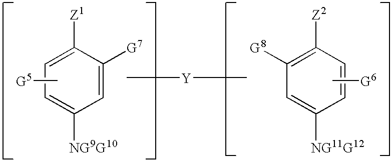
in which:
Z¹ and Z² denote, independently of one another, a hydroxyl or NH₂ radical that is optionally substituted with a C₁ to C₄ alkyl radical, with a C₁ to C₄ hydroxyalkyl radical, and/or with a bridge Y, or that optionally is part of a bridging ring system;
bridge Y denotes an alkylene group having 1 to 14 carbon atoms, for example a linear or branched alkylene chain or an alkylene ring, which can be interrupted or terminated by one or more nitrogen-containing groups and/or one or more heteroatoms such as oxygen, sulfur, or nitrogen atoms, and possibly can be substituted with one or more hydroxyl or C₁ to C₈ alkoxy radicals, or a direct bond;
G⁵ and G⁶ denote, independently of one another, a hydrogen or halogen atom, a C₁ to C₄ alkyl radical, a C₁ to C₄ monohydroxyalkyl radical, a C₂ to C₄ polyhydroxyalkyl radical, a C₁ to C₄ aminoalkyl radical, or a direct bond to bridge Y,
G⁷, G⁸, G⁹, G¹⁰, G¹¹ and G¹² denote, independently of one another, a hydrogen atom, a direct bond to bridge Y, or a C₁ to C₄ alkyl radical,
   with the provisos that
the compounds of formula (E2) contain only one bridge Y per molecule; and
the compounds of formula (E2) contain at least one amino group that carries at least one hydrogen atom.

The substituents used in formula (E2) are defined, according to the present invention, analogously to the statements made above.

Preferred binuclear developer components of formula (E2) are, in particular: N,N′-bis-(β-hydroxyethyl)-N,N′-bis-(4′-aminophenyl)-1,3-diaminopropan-2-ol, N,N′-bis-(β-hydroxyethyl)-N,N′-bis-(4′-aminophenyl)ethylenediamine, N,N′-bis-(4-aminophenyl)tetramethylenediamine, N,N′-bis-(β-hydroxyethyl)-N,N′-bis-(4-aminophenyl)-tetramethylenediamine, N,N′-bis-(4-methylaminophenyl)tetramethylenediamine, N,N′-diethyl-N,N′-bis-(4′-amino-3′-methylphenyl)ethylenediamine, bis-(2-hydroxy-5-aminophenyl)methane, N,N′-bis-(4′-aminophenyl)-1,4-diazacycloheptane, N,N′-bis-(2-hydroxy-5-aminobenzyl)piperazine, N-(4′-aminophenyl)-p-phenylenediamine, and 1,10-bis-(2′,5′-diaminophenyl)-1,4,7,10-tetraoxadecane, and their physiologically acceptable salts.

Very particularly preferred binuclear developer components of formula (E2) are N,N′-bis-(β-hydroxyethyl)-N,N′-bis-(4′-aminophenyl)-1,3-diaminopropan-2-ol, bis-(2-hydroxy-5-aminophenyl)methane, N,N′-bis-(4′-aminophenyl)-1,4-diazacycloheptane, and 1,10-bis-(2′,5′-diaminophenyl)-1,4,7,10-tetraoxadecane, or one of their physiologically acceptable salts.

It may furthermore be preferred according to the present invention to use as a developer component a p-aminophenol derivative or one of its physiologically acceptable salts. p-Aminophenol derivatives of formula (E3) are particularly preferred: 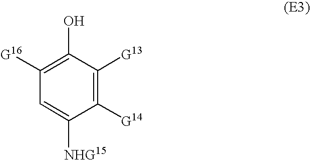
in which:
G¹³ denotes a hydrogen atom, a halogen atom, a C₁ to C₄ alkyl radical, a C₁ to C₄ monohydroxyalkyl radical, a C₂ to C₄ polyhydroxyalkyl radical, a (C₁ to C₄) alkoxy-(C₁ to C₄) alkyl radical, a C₁ to C₄ aminoalkyl radical, a hydroxy-(C₁ to C₄) alkylamino radical, a C₁ to C₄ hydroxyalkoxy radical, a C₁ to C₄ hydroxyalkyl-(C₁ to C₄) aminoalkyl radical, or a (di-C₁ to C₄ alkylamino)-(C₁ to C₄) alkyl radical, and
G¹⁴ denotes a hydrogen or halogen atom, a C₁ to C₄ alkyl radical, a C₁ to C₄ monohydroxyalkyl radical, a C₂ to C₄ polyhydroxyalkyl radical, a (C₁ to C₄) alkoxy-(C₁ to C₄) alkyl radical, a C₁- to C₄ aminoalkyl radical, or a C₁ to C₄ cyanoalkyl radical,
G¹⁵ denotes hydrogen, a C₁ to C₄ alkyl radical, a C₁ to C₄ monohydroxyalkyl radical, a C₂ to C₄ polyhydroxyalkyl radical, a phenyl radical, or a benzyl radical, and
G¹⁶ denotes hydrogen or a halogen atom.

The substituents used in formula (E3) are defined, according to the present invention, analogously to the statements made above.

Preferred p-aminophenols of formula (E3) are, in particular, p-aminophenol, N-methyl-p-aminophenol, 4-amino-3-methylphenol, 4-amino-3-fluorophenol, 2-hydroxymethylamino-4-aminophenol, 4-amino-3-hydroxymethylphenol, 4-amino-2-(-hydroxyethoxy)phenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-(α,β-dihydroxyethyl)phenol, 4-amino-2-fluorophenol, 4-amino-2-chlorophenol, 4-amino-2,6-dichlorophenol, 4-amino-2-(diethylaminomethyl)phenol, and their physiologically acceptable salts.

Very particularly preferred compounds of formula (E3) are p-aminophenol, 4-amino-3-methylphenol, 4-amino-2-aminomethylphenol, 4-amino-2-(α,β-dihydroxyethyl)phenol, and 4-amino-2-(diethylaminomethyl)phenol.

The developer component can furthermore be selected from o-aminophenol and its derivatives such as, for example, 2-amino-4-methylphenol, 2-amino-5-methylphenol, or 2-amino-4-chlorophenol.

The developer component can furthermore be selected from heterocyclic developer components such as, for example, the pyridine, pyrimidine, pyrazole, pyrazole-pyrimidine derivatives, and their physiologically acceptable salts.

Preferred pyridine derivatives are, in particular, the compounds that are described in British Patents GB 1 026 978 and GB 1 153 196, such as 2,5-diaminopyridine, 2-(4′-methoxyphenyl)amino-3-aminopyridine, 2,3-diamino-6-methoxypyridine, 2-(β-methoxyethyl)amino-3-amino-6-methoxypyridine, and 3,4-diaminopyridine.

Preferred pyrimidine derivatives are, in particular, the compounds described in German Patent DE 2 359 399, Japanese Unexamined Application JP 02019576 A2, or Unexamined Application WO 96/15765, for example 2,4,5,6-tetraaminopyrimidine, 4-hydroxy-2,5,6-triaminopyrimidine, 2-hydroxy-4,5,6-triaminopyrimidine, 2-dimethylamino-4,5,6-triaminopyrimidine, 2,4-dihydroxy-5,6-diaminopyrimidine, and 2,5,6-triaminopyrimidine.

Preferred pyrazole derivatives are, in particular, the compounds described in German Patents DE 3 843 892, DE 4 133 957, and Patent Applications WO 94/08969, WO 94/08970, EP 740 931, and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert.-butyl-1-methylpyrazole, 4,5-diamino-1-tert.-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, and 3,5-diamino-4-(β-hydroxyethyl)amino-1-methylpyrazole.

Preferred pyrazolopyrimidine derivatives are, in particular, the derivatives of pyrazolo[1,5-a]pyrimidine of formula (E4) below and its tautomeric forms, provided a tautomeric equilibrium exists: 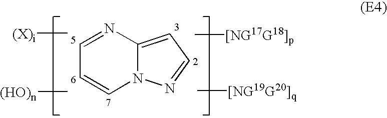
in which:
G¹⁷, G¹⁸, G¹⁹ and G²⁰ denote, independently of one another, a hydrogen atom, a C₁ to C₄ alkyl radical, an aryl radical, a C₁ to C₄ hydroxyalkyl radical, a C₂ to C₄ polyhydroxyalkyl radical, a (C₁ to C₄) alkoxy-(C₁ to C₄) alkyl radical, a C₁ to C₄ aminoalkyl radical that, if applicable, can be protected by an acetyl ureide or sulfonyl radical, a (C₁ to C₄) alkylamino-(C₁ to C₄) alkyl radical, a di-[(C₁ to C₄) alkyl]-(C₁ to C₄) aminoalkyl radical, the dialkyl radicals forming, if applicable, a carbon cycle or a heterocycle having five or six chain members, a C₁ to C₄ hydroxyalkyl radical, or a di-(C₁ to C₄) [hydroxyalkyl]-(C₁ to C₄) aminoalkyl radical;
the X radicals denote, independently of one another, a hydrogen atom, a C₁ to C₄ alkyl radical, an aryl radical, a C₁ to C₄ hydroxyalkyl radical, a C₂ to C₄ polyhydroxyalkyl radical, a C₁ to C₄ aminoalkyl radical, a (C₁ to C₄) alkylamino-(C₁ to C₄) alkyl radical, a di-[(C₁ to C₄) alkyl]-(C₁- to C₄) aminoalkyl radical, the dialkyl radicals forming, if applicable, a carbon cycle or a heterocycle having five or six chain members, a C₁ to C₄ hydroxyalkyl radical or a di-(C₁ to C₄ hydroxyalkyl)aminoalkyl radical, an amino radical, a C₁ to C₄ alkyl- or di-(C₁ to C₄ hydroxyalkyl)amino radical, a halogen atom, a carboxylic acid group, or a sulfonic acid group,
i has the value 0, 1, 2, or 3,
p has the value 0 or 1,
q has the value 0 or 1, and
n has the value 0 or 1,
   with the proviso that
the sum of p+q is not equal to 0,
if p+q is equal to 2, n has the value 0 and the groups NG¹⁷G¹⁸ and NG¹⁹G²⁰ occupy positions (2,3); (5,6); (6,7); (3,5) or (3,7);
if p+q is equal to 1, n has the value 1 and the groups NG¹⁷G¹⁸ (or NG¹⁹G²⁰) and the OH group occupy positions (2,3); (5,6); (6,7); (3,5) or (3,7);

The substituents used in formula (E4) are defined, according to the present invention, analogously to the statements made above.

If the pyrazolo[1,5-a]pyrimidine of the above formula (E4) contains a hydroxy group at one of positions 2, 5, or 7 of the ring system, a tautomeric equilibrium exists that is depicted, for example, in the following diagram:

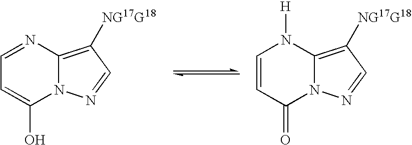

Among the pyrazolo[1,5-a]pyrimidines of the above formula (E4), the following may be mentioned in particular:
pyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
2-(7-aminopyrazolo[1,5-a]pyrimidin-3-ylamino)ethanol;
2-[(3-aminopyrazolo[1,5-a]pyrimidin-7-yl)(2-hydroxyethyl)amino]ethanol;
2-[(7-aminopyrazolo[1,5-a]pyrimidin-3-yl)(2-hydroxyethyl)amino]ethanol;
5,6-dimethylpyrazolo[1,5-a]pyrimidine-3,7-diamine;
2,6-dimethylpyrazolo[1,5-a]pyrimidine-3,7-diamine;
3-amino-7-dimethylamino-2,5-dimethylpyrazolo[1,5-a]pyrimidine;
   as well as their physiologically acceptable salts and their tautomeric forms, if a tautomeric equilibrium exists.

As described in the literature, the pyrazolo[1,5-a]pyrimidines of the above formula (E4) can be produced by cyclization proceeding from an aminopyrazole or from hydrazine.

Those indoles and indolines that comprise at least one hydroxy or amino group, preferably as a substituent on the six-membered ring, are preferred for use as precursors of nature-analogous dyes. These groups can carry further substituents, e.g., in the form of an etherification or esterification of the hydroxy group or an alkylation of the amino group. In a second preferred embodiment, the coloring agents contain at least one indole derivative and/or indoline derivative.

Derivatives of 5,6-dihydroxyindoline of formula (IIa) are particularly suitable, as developer components, as precursors of nature-analogous hair dyes: 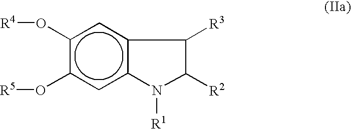
in which, independently of one another:
R¹ denotes hydrogen, a C₁-C₄ alkyl group, or a C₁-C₄ hydroxyalkyl group,
R² denotes hydrogen or a —COOH group, such that the —COOH group can also be present as a salt having a physiologically acceptable cation,
R³ denotes hydrogen or a C₁-C₄ alkyl group,
R⁴ denotes hydrogen, a C₁-C₄ alkyl group, or a —CO—R⁶ group in which R⁶ denotes a C₁-C₄ alkyl group, and
R⁵ denotes one of the groups listed under R⁴,
as well as physiologically acceptable salts of these compounds with an organic or inorganic acid.

Particularly preferred derivatives of indoline are 5,6-dihydroxyindoline, N-methyl-5,6-dihydroxyindoline, N-ethyl-5,6-dihydroxyindoline, N-propyl-5,6-dihydroxyindoline, N-butyl-5,6-dihydroxyindoline, 5,6-dihydroxyindoline-2-carboxylic acid, as well as 6-hydroxyindoline, 6-aminoindoline, and 4-aminoindoline.

Particularly to be emphasized within this group are N-methyl-5,6-dihydroxyindoline, N-ethyl-5,6-dihydroxyindoline, N-propyl-5,6-dihydroxyindoline, N-butyl-5,6-dihydroxyindoline, and in particular 5,6-dihydroxyindoline.

Derivatives of 5,6-dihydroxyindole of formula (IIb) are furthermore outstandingly suitable as precursors of nature-analogous hair dyes: 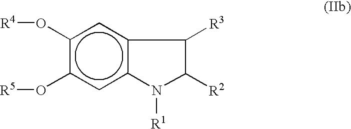
in which, independently of one another:
R¹ denotes hydrogen, a C₁-C₄ alkyl group, or a C₁-C₄ hydroxyalkyl group,
R² denotes hydrogen or a —COOH group, such that the —COOH group can also be present as a salt having a physiologically acceptable cation,
R³ denotes hydrogen or a C₁-C₄ alkyl group,
R⁴ denotes hydrogen, a C₁-C₄ alkyl group, or a —CO—R⁶ group in which R⁶ denotes a C₁-C₄ alkyl group, and
R⁵ denotes one of the groups listed under R⁴,
   as well as physiologically acceptable salts of these compounds with an organic or inorganic acid.

Particularly preferred derivatives of indole are 5,6-dihydroxyindole, N-methyl-5,6-dihydroxyindole, N-ethyl-5,6-dihydroxyindole, N-propyl-5,6-dihydroxyindole, N-butyl-5,6-dihydroxyindole, 5,6-dihydroxyindote-2-carboxylic acid, and 6-hydroxyindole, 6-aminoindole, and 4-aminoindole.

To be emphasized within this group are N-methyl-5,6-dihydroxyindole, N-ethyl-5,6-dihydroxyindole, N-propyl-5,6-dihydroxyindole, N-butyl-5,6-dihydroxyindole, and in particular 5,6-dihydroxyindole.

The indoline and indole derivatives can be used in compositions (A) and (B) according to the present invention both as free bases and in the form of their physiologically acceptable salts with inorganic or organic acids, e.g., the hydrochlorides, sulfates, and hydrobromides. The indole or indoline derivatives are contained therein usually in quantities from 0.05 to 10 wt %, preferably 0,2 to 5 wt %.

In a further embodiment, provision may be made according to the present invention for the indoline or indole derivative to be used in combination with at least one amino acid or one oligopeptide. The amino acid is advantageously an (x-amino acid; very particularly preferred a-amino acids are arginine, ornithine, lysine, serine, and histidine, in particular arginine.

In a further embodiment, compositions (A) or (B) according to the present invention contain at least one coupler component.

m-Phenylenediamine derivatives, naphthols, resorcinol and resorcinol derivatives, pyrazolones, and m-aminophenol derivatives are generally used as coupler components. 1-Naphthol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methylpyrazolone-5,2,4-dichloro-3-aminophenol, 1,3-bis-(2′,4′-diaminophenoxy)propane, 2-chlororesorcinol, 4-chlororesorcinol, 2-chloro-6-methyl-3-aminophenol, 2-amino-3-hydroxypyridine, 2-methylresorcinol, 5-methylresorcinol, and 2-methyl-4-chloro-5-aminophenol are particularly suitable as coupler substances.

Coupler components preferred according to the present invention are
m-aminophenol and its derivatives such as, for example, 5-amino-2-methylphenol, N-cyclopentyl-3-aminophenol, 3-amino-2-chloro-6-methylphenol, 2-hydroxy-4-aminophenoxyethanol, 2,6-dimethyl-3-aminophenol, 3-trifluoroacetylamino-2-chloro-6-methylphenol, 5-amino-4-chloro-2-methylphenol, 5-amino-4-methoxy-2-methylphenol, 5-(2′-hydroxyethyl)amino-2-methylphenol, 3-(diethylamino)phenol, N-cyclopentyl-3-aminophenol, 1,3-dihydroxy-5-(methylamino)benzene, 3-ethylamino-4-methylphenol, and 2,4-dichloro-3-aminophenol,
o-aminophenol and its derivatives,
m-diaminobenzene and its derivatives such as, for example, 2,4-diaminophenoxyethanol, 1,3-bis-(2′,4′-diaminophenoxy)propane, 1-methoxy-2-amino-4-(2′-hydroxyethylamino)benzene, 1,3-bis-(2′,4′-diaminophenyl)propane, 2,6-bis-(2′-hydroxyethylamino)-1-methylbenzene, and 1-amino-3-bis-(2′-hydroxyethyl)aminobenzene,
o-diaminobenzene and its derivatives such as, for example, 3,4-diaminobenzoic acid and 2,3-diamino-1-methylbenzene,
di- and trihydroxybenzene derivatives such as, for example, resorcinol, resorcinol monomethyl ether, 2-methylresorcinol, 5-methylresorcinol, 2,5-dimethylresorcinol, 2-chlororesorcinol, 4-chlororesorcinol, pyrogallol, and 1,2,4-trihydroxybenzene, pyridine derivatives such as, for example, 2,6-dihydroxypyridine, 2-amino-3-hydroxypyridine, 2-amino-5-chloro-3-hydroxypyridine, 3-amino-2-methylamino-6-methoxypyridine, 2,6-dihydroxy-3,4-dimethylpyridine, 2,6-dihydroxy-4-methylpyridine, 2,6-diaminopyridine, 2,3-diamino-6-methoxypyridine and 3,5-diamino-2,6-dimethoxypyridine,
naphthalene derivatives such as, for example, 1-naphthol, 2-methyl-1-naphthol, 2-hydroxymethyl-1-naphthol, 2-hydroxyethyl-1-naphthol, 1,5-dihydroxynaphthalene, 1,6-dihydroxynaphthalene, 1,7-dihydroxynaphthalene, 1,8-dihydroxynaphthalene, 2,7-dihydroxynaphthalene, and 2,3-dihydroxynaphthalene,
morpholine derivatives such as, for example, 6-hydroxybenzomorpholine and 6-aminobenzomorpholine,
quinoxaline derivatives such as, for example, 6-methyl-1,2,3,4-tetrahydroquinoxaline,
pyrazole derivatives such as, for example, 1-phenyl-3-methylpyrazol-5-one,
indole derivatives such as, for example, 4-hydroxyindole, 6-hydroxyindole, and 7-hydroxyindole,
pyrimidine derivatives such as, for example, 4,6-diaminopyrimidine, 4-amino-2,6-dihydroxypyrimidine, 2,4-diamino-6-hydroxypyrimidine, 2,4,6-trihydroxypyrimidine, 2-amino-4-methylpyrimidine, 2-amino-4-hydroxy-6-methylpyrimidine, and 4,6-dihydroxy-2-methylpyrimidine, or
methylenedioxybenzene derivatives such as, for example, 1-hydroxy-3,4-methylenedioxybenzene, 1-amino-3,4-methylenedioxybenzene, and 1-(2′-hydroxyethyl)amino-3,4-methylenedioxybenzene.

Coupler components particularly preferred according to the present invention are 1-naphthol, 1,5-, 2,7- and 1,7-dihydroxynaphthalene, 3-aminophenol, 5-amino-2-methylphenol, 2-amino-3-hydroxypyridine, resorcinol, 4-chlororesorcinol, 2-chloro-6-methyl-3-aminophenol, 2-methylresorcinol, 5-methylresorcinol, 2,5-dimethylresorcinol, and 2,6-dihydroxy-3,4-dimethylpyridine.

It is preferred that, in the embodiment as a coloring agent, one of compositions (A) and (B) contain no hydrogen peroxide, and that the direct-absorbing dyes or the oxidizing dye precursors be contained in that hydrogen peroxide-free composition.

Compositions (A) and/or (B) according to the present invention can furthermore contain all ingredients, additives, and adjuvants known for such preparations.

Further ingredients, adjuvants, and additives are, for example:
nonionic polymers such as, for example, vinylpyrrolidone/vinyl acrylate copolymers, polyvinylpyrrolidone, and vinylpyrrolidone/vinyl acetate copolymers, and polysiloxanes,
zwitterionic and amphoteric polymers such as, for example, acrylamidopropyltrimethylammonium chloride/acrylate copolymers and octylacrylamide/methyl methacrylate/tert-butylaminoethyl methacrylate/2-hydroxypropyl methacrylate copolymers,
anionic polymers such as, for example, polyacrylic acids, crosslinked polyacrylic acids, vinyl acetate/crotonic acid copolymers, vinylpyrrolidone/vinyl acrylate copolymers, vinyl acetate/butyl maleate/isobornyl acrylate copolymers, methyl vinyl ether/maleic acid anhydride copolymers, and acrylic acid/ethyl acrylate/N-tert.butylacrylamide terpolymers,
thickening agents such as agar-agar, guar gum, alginates, xanthan gum, gum arabic, karaya gum, locust bean flour, linseed gums, dextrans, cellulose derivatives, e.g., methyl cellulose, hydroxyalkyl cellulose, and carboxymethyl cellulose, starch fractions and derivatives such as amylose, amylopectin, and dextrins, clays such as, e.g., bentonite, or entirely synthetic hydrocolloids such as, e.g., poly(vinyl alcohol),
structuring agents such as maleic acid and lactic acid,
protein hydrolysates, in particular elastin, collagen, keratin, milk-protein, soy-protein and wheat-protein hydrolysates, their condensation products with fatty acids, and quaternized protein hydrolysates,
perfume oils, dimethyl isosorbide, and cyclodextrins,
solvents and solubilizers such as ethanol, isopropanol, ethylene glycol, propylene glycol, glycerol, and diethylene glycol,
quaternized amines such as methyl-1-alkylamidoethyl-2-alkylimidazolinium methosulfate,
defoamers such as silicones,
anti-dandruff ingredients such as piroctone olamine, zinc omadine, and climbazol,
light protection agents, in particular derivatized benzophenones, cinnamic acid derivatives, and triazines,
substances for adjusting pH, such as, for example, usual acids, in particular edible acids and bases,
ingredients such as allantoin, pyrrolidonecarboxylic acids, and their salts, as well as bisabolol,
cholesterol,
consistency agents such as sugar esters, polyol esters, or polyol alkyl ethers,
fats and waxes such as spermaceti, beeswax, montan wax, and paraffins,
fatty acid alkanolamides,
swelling and penetrating substances such as glycerol, propylene glycol monoethyl ether, carbonates, hydrogencarbonates, guanidine, ureas, as well as primary, secondary, and tertiary phosphates,
opacifiers such as latex, styrene/PVP and styrene/acrylamide copolymers
luster agents such as ethylene glycol mono- and distearate, as well as PEG-3 distearate,
pigments,
stabilizing agents for hydrogen peroxide and other oxidizing agents,
propellants such as propane-butane mixtures, N₂O, dimethyl ether, CO₂, and air,
antioxidants.

With regard to further optional components as well as the quantities of those components used, the reader is referred expressly to the relevant manuals known to those skilled in the art, e.g., Kh. Schrader, Grundlagen und Rezepturen der Kosmetika, [Cosmetics fundamentals and formulas], 2nd edition, Huthig Buch Verlag, Heidelberg, 1989.

If composition (A) is present as a solid, composition (A) can then be manufactured in the form of a powder or a shaped element (i.e., a granulate, extrudate, or pressed item, e.g., in the shape of a tablet).

Production of the shaped elements according to the present invention is accomplished firstly by dry mixing of the constituents, which can be entirely or partly pre-granulated, and by subsequent shaping, in particular compression into tablets, in which context known methods can be resorted to. For production of the shaped elements according to the present invention, the premix is compacted in a so-called mold between two dies, yielding a solid compressed body. This operation, which will be referred to hereinafter for brevity's sake as tableting, is subdivided into four portions: metering, compaction (elastic deformation), plastic deformation, and ejection.

Firstly the premix is introduced into the mold, the fill quantity and therefore the weight and the shape of the resulting shaped element being determined by the position of the lower die and the shape of the pressing tool. Consistent metering even at high shaped-element throughput rates is preferably achieved by volumetric metering of the premix. As tableting proceeds, the upper die comes into contact with the premix and moves farther downward toward the lower die. This compaction causes the particles of the premix to be pressed closer to one another, while the cavity volume inside the filled material between the dies continuously decreases. Beyond a certain position of the upper die (and therefore above a certain pressure on the premix), plastic deformation begins, in which the particles merge together and formation of the shaped element occurs. Depending on the physical properties of the premix, some of the premix particles are also crushed, and at even higher pressures a sintering of the premix occurs. As the pressing speed rises, i.e. at high throughput rates, the elastic deformation phase becomes increasingly shorter, so that the resulting shaped elements may exhibit cavities of varying sizes. In the last phase of tableting, the completed shaped elements are pushed out of the mold by the lower die, and are carried away by downstream transport devices. At this point in time only the weight of the shaped element is completely defined, since physical processes (rebound, crystallographic effects, cooling, etc.) can still cause the shape and size of the compacts to change.

Tableting is performed in commercially available tableting presses that can be equipped in principle with single or double dies. In the latter case only the upper die is used to build up pressure; the lower die also moves toward the upper die during the pressing process, while the upper die pushes downward. For small production volumes it is preferred to use eccentric tableting presses in which the die or dies are attached to an eccentric disk that in turn is mounted on a shaft having a specific rotation speed. The movement of these pressing dies is comparable to the manner of operation of a conventional four-stroke engine. Pressing can be accomplished using one upper and one lower die, but multiple dies can also be attached to one eccentric disk, the number of mold orifices being correspondingly increased. The throughput rates of eccentric presses vary, depending on type, from a few hundred to a maximum of 3,000 tablets per hour.

Rotary tablet presses, in which a larger number of molds is arranged in a circle on a so-called mold table, are selected for higher throughput rates. The number of molds varies, depending on the model, from six to 55, larger molds also being commercially available. Each mold on the mold table has an upper and a lower die associated with it; once again the applied pressure can be actively built up only by the upper or lower die, but also by both dies. The mold table and the dies move about a common vertically oriented axis, the dies being brought during rotation, with the aid of rail-like curved tracks, into the positions for filling, compaction, plastic deformation, and ejection. At the points where a particularly pronounced raising or lowering of the dies is necessary (filling, compaction, ejection), these curved tracks are assisted by additional press-down elements, press-down rails, and lifting tracks. The molds are filled via a rigidly arranged delivery device called the filling shoe, which is connected to a reservoir for the premix. The applied pressure on the premix is individually adjustable by way of the pressing travels for the upper and lower dies, pressure being built up as the die shaft heads roll past displaceable pressure rollers.

To increase the throughput rate, rotary presses can also be equipped with two filling shoes, in which case only a half-circle rotation is necessary in order to produce a tablet. For the production of two-layer and multi-layer shaped elements, multiple filling shoes are arranged one behind the other, and the slightly compressed first layer is not ejected before further filling. With appropriate process control, it is possible in this fashion also to produce coated tablets and core tablets that have an onion-like structure; in the case of core tablets, the top of the core or of the core layers is not covered and thus remains visible. Rotary tableting presses can also be fitted with single or multiple molds so that, for example, an outer circle having 50 orifices and an inner circle having 35 orifices can be used simultaneously for pressing. The throughput rates of modern rotary tableting presses are more than a million shaped elements per hour.

In the context of tableting with rotary presses, it has proven advantageous to perform tableting with the smallest possible fluctuations in tablet weight. This also allows fluctuations in tablet hardness to be reduced. Small weight fluctuations can be achieved in the following fashion:
use of plastic inserts having small thickness tolerances
low rotor rotation speed
large filling shoes
coordination between filling shoe blade speed and rotor rotation speed
constant powder height in the filling shoe
decoupling of filling shoe and powder supply.

All anti-adhesion coatings known in the art are suitable for reducing die caking. Plastic coatings, plastic inserts, or plastic dies are particularly advantageous. Rotating dies have also proven advantageous; if possible, the upper and lower dies should be configured rotatably. A plastic insert can usually be dispensed with in the case of rotating dies. In this case the die surfaces should be electropolished.

It has furthermore become apparent that long pressing times are advantageous. These can be using pressing rails, multiple pressing rollers, or low rotor rotation speeds. Because fluctuations in tablet hardness can be caused by fluctuations in pressing forces, systems that limit the pressing force should be utilized. Elastic dies, pneumatic compensators, or resilient elements in the force path can be used here. The pressing roller can also be embodied resiliently.

Tableting machines that are suitable in the context of the present invention are obtainable, for example, from the following companies: Apparatebau Holzwarth GbR, Asperg; Wilhelm Felte GmbH, Schwarzenbek; Fann Instruments Company, Houston, Tex. (USA); Hofer GmbH, Weil; Horn & Noack Pharmatechnik GmbH, Worms; IMA Verpackungssysteme GmbH Viersen; KILIAN, Cologne; KOMAGE, Kell am See; KORSCH Pressen AG, Berlin; and Romaco GmbH, Worms. Additional suppliers are, for example, Dr. Herbert Pete, Vienna (AT); Mapag Maschinenbau AG, Bern (CH); BWI Manesty, Liverpool (GB); I. Holand Ltd., Nottingham (GB); Courtoy N.V., Halle (BE/LU); and Mediopharm Kamnik (SI). The HPF 630 hydraulic double-pressure press of the LAEIS company (D) is, for example, particularly suitable. Tableting tools are available, for example, from the following companies: Adams Tablettierwerkzeuge, Dresden; Wilhelm Fett GmbH, Schwarzenbek; Klaus Hammer, Solingen; Herber & Söhne GmbH, Hamburg; Hofer GmbH, Weil; Horn & Noack, Pharmatechnik GmbH, Worms; Ritter Pharamatechnik GmbH, Hamburg; Romaco, GmbH, Worms; and Notter Werkzeugbau, Tamm. Additional suppliers are, for example, Senss AG, Reinach (CH) and Medicopharm, Kamnik (SI).

The method for producing the shaped elements is not, however, limited to pressing only one particulate premix into a shaped element. The method can instead also be expanded in that multi-layer shaped elements are produced in known fashion, by preparing two or more premixes that are pressed onto one another. The premix that is charged first is slightly precompressed in order to create a smooth upper surface extending parallel to the base of the shaped element, and is finally compressed, after addition of the second premix, to yield the completed shaped element. In the case of three- or multi-layer shaped elements, a further precompression occurs after each addition of premix, before the shaped element is finally compressed after addition of the last premix.

A third subject of the invention is an anhydrous composition that contains at least one alkalization-effecting solid composition of the first subject of the invention, as well as at least one bleach booster.

The alkalization-effecting solid composition according to the present invention is contained in the anhydrous composition preferably in a quantity from 1 to 40 wt %, particularly preferably in a quantity from 2 to 30 wt %, in each case based on the weight of the entire anhydrous composition.

Regarding the term “bleach booster” and its preferred representatives, the statements made in this context with respect to the second subject of the invention are applicable. The bleach boosters are present preferably in a quantity from 5 to 60 wt %, in particular in quantities from 8 to 30 wt %, based on the entire anhydrous composition.

The anhydrous composition can optionally contain hydrogen peroxide in the form of addition products of hydrogen peroxide with solid organic or inorganic compounds. The preferred representatives of these addition products have already been summarized in the second subject of the invention.

The anhydrous composition according to the present invention is preferably solid or pasty. The statements made in the second subject of the invention are applicable to these embodiments.

The anhydrous composition according to the present invention can additionally contain at least one compound selected from surfactants, conditioning ingredients, structure-improving ingredients, and ingredients, adjuvants, and additives in preferred quantities. The statements made in the second subject of the invention are applicable to these embodiments.

The anhydrous composition according to the present invention can additionally contain at least one compound from the group of the developer components, coupler components, direct-absorbing dyes, and precursors of nature-analogous dyes. The preferred representatives of these compounds have already been summarized in the second subject of the invention.

A fourth subject of the present invention is a method for stabilizing H₂O₂ during the dissolution process of a solid or pasty composition (A) in an aqueous or aqueous/alcoholic composition (B), composition (A) containing, as an alkalizing agent, at least one alkalization-effecting solid composition of the first subject of the invention; and at least one of compositions (A) and (B) containing hydrogen peroxide.

It is preferred according to the present invention that only composition (B) contain hydrogen peroxide.

Regarding the features and preferred embodiments of compositions (A) and (B), the statements made regarding the hydrogen peroxide-containing agents and their compositions (A) and (B) of the second subject of the invention are applicable.

Regarding the features and preferred embodiments of the alkalization-effecting solid composition, the statements made regarding the composition of the first subject are applicable.

A fifth subject of the present invention is a method for brightening keratin-containing fibers in which the fibers are treated with one of the above-described hydrogen peroxide-containing agents of the second subject of the invention.

A sixth subject of the present invention is a method for coloring keratin-containing fibers in which the fibers are treated with a hydrogen peroxide-containing agent of the second subject of the invention, in the presence of direct-absorbing dyes and/or oxidizing dye precursors.

A seventh subject of the present invention is a method for cleaning textiles and hard surfaces in which the textiles or hard surfaces are treated with one of the above-described hydrogen peroxide-containing agents of the second subject of the invention.

The examples that follow are intended to explain the subject matter of the present invention without, however, limiting it.

### EXAMPLES

1.0 Production Of An Agglomerate 1 According To The Present Invention 56.0 g Britesil° C20¹ and 121 g water were mixed. 0.4 g Turpinal® SL² was dripped into this mixture while stirring. The mixture was dried in a rotary evaporator (125 rpm) at 80° C. in vacuum. The mixture was placed in a beaker and dried for a further three days in a desiccating cabinet at 50° C.
¹ Sodium silicate (INCI name: Sodium silicate) (The PQ Corporation)

² 1-Hydroxyethane-1,1-diphosphonic acid (60% active substance in water) (INCI name: Etidronic acid, Aqua (water)) (Solutia)

2.0 Example of Use in Hair-Bleaching Powders

Hair-bleaching powder 1 (according to present invention)

| | |
|---|---|
| Ammonium peroxodisulfate | 13.00 g |
| Potassium persulfate | 42.80 g |
| Sodium persulfate | 15.60 g |
| Agglomerate 1 (per 1.0) | 28.20 g |
| Idranal ® III³ | 0.60 g |

¹Ethylenediaminetetraacetic acid disodium salt.2H₂O (INCI name: Disodium EDTA) (Manufacturer: Riedel De Haen)

3.0 Comparative Tests

For comparison with hair-bleaching powder 1 according to the present invention, the following hair-bleaching powders not according to the present invention were prepared:

| Hair-bleaching powder V1 | | Hair-bleaching powder V2 | |
|---|---|---|---|
| Ammonium | 13.00 g | Ammonium | 13.00 g |
| peroxodisulfate | | peroxodisulfate | |
| Potassium persulfate | 42.80 g | Potassium persulfate | 42.80 g |
| Sodium persulfate | 15.60 g | Sodium persulfate | 15.60 g |
| Britesil ® C20 | 28.00 g | Britesil ® C20 | 28.00 g |
| Idranal ® III | 0.60 g | Idranal ® III | 0.60 g |
| | | Etidronic acid (solid) | 2.00 g |

Hair-bleaching powder 1 contained 28.02 wt % alkalizing agent in the form of Britesil® C20 in agglomerate 1 and, as chelating agent, 0.12 wt % etidronic acid from agglomerate 1, as well as 0.60 wt % EDTA disodium salt.

Hair-bleaching powder V1 contained 28.00 wt % alkalizing agent in the form of Britesil® C20, and 0.60 wt % EDTA disodium salt as a chelating agent.

Hair-bleaching powder V2 contained 27.45 wt % alkalizing agent in the form of Britesil® C20 and, as chelating agent, 0.59 wt % EDTA disodium salt, and 1.96 wt % etidronic acid.

3.1 Testing Decomposition of Hydrogen Peroxide During the Mixing Process

In the tests below, the corresponding hydrogen peroxide-containing creme of the product Poly® Intensiv Aufheller Ultra Plus (Schwarzkopf-Henkel) was used as the hydrogen peroxide-containing creme. The storage container of the hydrogen peroxide-containing creme of the commercial product was used as the bottle.

3.1.1 Experiment 1

20.0 g of each of the hair-bleaching powder formulations (1, V1, or V2) was placed in a respective bottle with 50.0 g of a hydrogen peroxide-containing creme, and the bottle was closed. The bottle was shaken for 30 seconds. After a 20-second resting period, the upright bottle was then opened and the elapsed time (emergence time) until the bottle contents began to emerge from the bottle due to gas evolved in the decomposition reaction was recorded.

3.1.2 Experiment

220.0 g of each of the hair-bleaching powder formulations (1, V1, or V2) was placed in a respective bottle with 50.0 g of a hydrogen peroxide-containing creme, and the bottle was closed. The bottle was shaken for 30 seconds The closed bottle was left to stand for a further 30 minutes. After this resting period the bottle was opened, and the behavior of the system during opening was observed.

3.1.3 Experimental Observations

Hair-Bleaching Powder 1 (according to present invention)

Experiment 1: Emergence time: 1 hour 55 minutes

Experiment 2: Fizzed slightly when bottle was opened. No product emergence occurred during or immediately after opening of the bottle.

Hair-Bleaching Powder V1

Experiment 1: Emergence time: 29 minutes

Experiment 2: Fizzed strongly and sprayed out when bottle was opened. A vigorous fountain-like product emergence occurred immediately after bottle was opened.

Hair-Bleaching Powder V2

Experiment 1 :Emergence time: 38 minutes

Experiment 2: Fizzed strongly and sprayed out when bottle was opened. No product emergence occurred during or immediately after opening of the bottle

Although hair-bleaching powder V2 contained more chelating agent (2.55 wt % total) than hair-bleaching powder 1 according to the present invention (0.72 wt % total), after the process of mixing hair-bleaching powder V2 with the hydrogen peroxide-containing creme, a considerable hydrogen peroxide decomposition reaction, and a resulting gas evolution, took place.

1. An alkalization-effecting composition comprising
(i) at least 75 wt % based on the weight of the composition, of a mixture of (a) at least one particulate alkalizing agent and (b) at least one chelating agent; and
(ii) optionally further additives, the composition comprising agglomerates formed from (a), (b) and the optional additives.

2. The composition according to claim 1, wherein the particulate alkalizing agent is selected from the group consisting of hydroxides, carbonates, hydrogencarbonates, hydroxycarbonates, carbamides, silicates (in particular metasilicates) of ammonium, of alkali metals, and of alkaline-earth metals and alkaline phosphates.

3. The composition according to claim 1, wherein at least one optionally hydrated silicate of the formula (SiO₂)ₙ(Na₂O)ₘ(K₂O)ₚ, is present as a particulate alkalizing agent, where n denotes a positive whole number and m and p, independently of one another, denote a positive whole number or 0, with the provisos that at least one of the parameters m or p is different from 0, and that the ratio between n and the sum of m and p is between 1:4 and 4:1.

4. The composition according to claim 1, wherein the chelating agent is selected from the group consisting of ethylenediaminetetraacetic acid, diethylenetriaminepentaacetic acid, nitriloacetic acid, 1-hydroxyethane-1,1-diphosphonic acid (etidronic acid), dipicolinic acid, acetanilide, and sodium stannate, and the physiologically acceptable salts of the aforesaid acids.

5. The composition according to claim 1, wherein the particulate alkalizing agents are present in a quantity of from 80 to 99.8 wt %, based on the entire composition.

6. The composition according to claim 1, wherein the chelating agents are present in a quantity from 0.1 to 20 wt %, based on the entire composition.

7. The composition according to claim 1, wherein the agglomerates have an average particle diameter from 10 to 300 μm.

8. The composition according to claim 1, wherein it exists in the form of a powder, extrudate, or granulate.

9. A hydrogen peroxide-containing agent that is obtained by mixing at least two separately manufactured compositions (A) and (B), wherein composition (A) is anhydrous and comprises at least one alkalization-effecting solid composition comprising (i) at least 75 wt % based on the weight of the composition of a mixture of (a) at least one particulate alkalizing agent and (b) at least one chelating agent and (ii) optionally further additives, the composition comprising agglomerates formed from (a), (b) and the optional additives, composition (B) is aqueous or aqueous/alcoholic; and at least one of compositions (A) and (B) comprises hydrogen peroxide.

10. The agent according to claim 9, wherein composition.(a) additionally comprises at least one bleach booster.

11. The agent according to claim 10, wherein the bleach booster is selected from the group consisting of (i) peroxodisulfates, persulfates, and perphosphates of ammonium and of the alkaline and alkaline-earth metals, (ii) peroxides of the alkali and alkaline-earth metals, (iii) peroxocarboxylic acids and their physiologically acceptable salts, and (iv) cationic nitrites.

12. The agent according to claim 10, wherein the bleach booster is selected from the group consisting of ammonium peroxodisulfate, potassium peroxodisulfate, sodium peroxodisulfate, ammonium persulfate, potassium persulfate, sodium persulfate, potassium peroxodiphosphate, magnesium perphthalate, magnesium peroxide and barium peroxide.

13. The agent according to claim 9, wherein composition (B) possesses a viscosity from 1 to 100,000 mPa s (Brookfield RVT viscosimeter, temperature 20° C., no. 4 spindle, 4 rpm).

14. The agent according to claim 9, wherein composition (B) has a viscosity from 6,000 to 50,000 mPa s (Brookfield RVT viscosimeter, temperature 20° C., no. 4 spindle, 4 rpm).

15. The agent according to claim 9, wherein the pH of composition (B) is in a range from pH 2 to 7.

16. The agent according to claim 9, wherein the agent has a pH greater than 7.

17. The agent according to claim 9, wherein it additionally comprises at least one nonionic, anionic, cationic, zwitterionic, or amphoteric surfactant.

18. The agent according to claim 9, wherein it additionally comprises at least one conditioning ingredient.

19. The agent according to claim 9, wherein it additionally comprises at least one structure-improving ingredient.

20. The agent according to claim 9, wherein it additionally comprises at least one direct-absorbing dye and/or at least one oxidizing dye precursor of the developer or coupler type.

21. The anhydrous composition comprising an alkalization-effecting solid composition according to claim 1 and at least one bleach booster.

22. A method for stabilizing H₂O₂ during the dissolution process of a solid or pasty composition (A) in an aqueous or aqueous/alcoholic composition (B), composition (A) comprising an alkalinization-effecting solid composition comprising
(i) at least 75 wt % based on the weight of the composition, of a mixture of (a) at least one particulate alkalizing agent and (b) at least one chelating agent; and
(ii) optionally further additives,
the composition comprising agglomerates formed from (a), (b) and the optional additives and at least one of compositions (A) and (B) comprising hydrogen peroxide.

23. The method for brightening keratin-containing fibers in which the fibers are treated with a hydrogen peroxide-containing agent according to claim 9.

24. The method for coloring keratin-containing fibers in which the fibers are treated with a hydrogen peroxide-containing agent according to claim 20.

25. The method for cleaning textiles and hard surfaces in which the textiles or hard surfaces are treated with a hydrogen peroxide-containing agent according to claim 9.
